(19) United States
(12) Patent Application Publication
(10) Pub. No.: US 2015/0376638 A1
(43) Pub. Date: Dec. 31, 2015

(54) BREEDING METHODS FOR ENHANCED GRAIN YIELD AND RELATED MATERIALS AND METHODS

(71) Applicant: INTERNATIONAL RICE RESEARCH INSTITUTE, Metro Manila (PH)

(72) Inventors: Tsutomu ISHIMARU, Los Banos, Laguna (PH); Inez Hortense SLAMET-LOEDIN, Los Banos, Laguna (PH); Daisuke FUJITA, Saga (JP); Kurniawan Rudi TRIJATMIKO, Bogor (ID); Yohei KOIDE, Kyoto-shi, Kyoto-fu (JP); Kazuhiro SASAKI, Tokyo (JP); Nikolaos K/ TSAKIRPALOGLOU, Los Banos, Laguna (PH); Yoshimichi FUKUTA, Ishigaki (JP); Nobuya KOBAYASHI, Tsukuba (JP)

(73) Assignee: Japann International Research Center Agriculturall Sciences, Tsukuba (JP)

(21) Appl. No.: 14/765,339

(22) PCT Filed: Feb. 3, 2014
(86) PCT No.: PCT/IB2014/000607

§ 371 Date: Aug. 2, 2015

Related U.S. Application Data
(60) Provisional application No. 61/759,408, filed on Feb. 1, 2013.

(51) Int. Cl. C12N 15/82; A01H 5/10; C12Q 1/68; A01H 1/02

(52) U.S. Cl. CPC C12N 15/8261; A01H 1/02; A01H 5/10; C12Q 1/6895; C12Q 2600/13

(57) ABSTRACT

Described herein are breeding methods useful to increase grain yield. Disclosed is a novel gene, SPIKE, which is shown herein to increase grain yield of modern indica cultivars and can be used to assist development of improved grains. Also described herein are materials and methods for increasing the grain yield of modern indica cultivars.

### CROSS-REFERENCE TO RELATED APPLICATIONS

This application claims priority to U.S. Provisional Application No. 61/759,408, filed on Feb. 1, 2013, the entire disclosure of which is expressly incorporated herein by reference for all purposes.

### SEQUENCE LISTING

The instant application contains a Sequence Listing which has been submitted via EFS-web and is hereby incorporated by reference in its entirety. The ASCII copy, created on Feb. 3, 2014, is named 53-55557-IRRI-13-002_SL, and is 103,543 bytes in size.

### BACKGROUND OF THE INVENTION

The world's population is expected to surpass 9 billion in 2050. Most of this increase will occur in the developing countries of Asia and Africa. By 2035, a 26% increase in rice production will be essential to feed the rising population. Although the Green Revolution led to increased grain production in the 1960s, no major advances have been made in increasing yield potential in rice since then.

Rice (Oryza sativa L.) is a staple food of more than 3 billion people, mainly in Asia. Indica cultivars are grown in southern China, Southeast Asia, and South Asia, occupying about 70% of the rice-producing area in the world, while japonica cultivars are grown mainly in East Asia. Because of urbanization and industrialization, an increase in the yield of indica cultivars is a pressing need in Southeast and South Asia. Additionally, good grain quality (influencing market value) and high yield are essential for the adoption of new cultivars in local areas.

The grain yield of rice is determined by spikelet number per panicle, panicle number per plant, grain weight, and spikelet fertility. Although many quantitative trait loci (QTLs) for yield components have been identified, few have so far been isolated. To date, at least nine genes or loci for yield-related traits in rice have been isolated from natural variation: Gn1a and APO1 for number of grains; GS3, GW2, and qSW5 for grain size; DEP1 and WFP for panicle architecture; SCM2 for strong culm; and Ghd7 for late heading and number of grains. APO1, SCM2, and DEP1 increased grain yield in a japonica genetic background in field experiments. However, no novel cloned gene has been reported to increase grain yield in indica cultivars.

Identification of a gene capable of increasing grain yield in indica cultivars is necessary in order to generate higher-yielding cultivars, thus helping to meet the increasing demand for rice production.

### SUMMARY OF THE INVENTION

The present invention provides methods for producing a progeny rice plant having improved grain yield comprising: providing a first rice plant comprising a gene SPIKE; crossing the first rice plant with a second rice plant to produce progeny rice plants; analyzing the second rice plant for the gene SPIKE; identifying and selecting progeny rice plants comprising the gene SPIKE and having improved grain yield over the second rice plant.

Also provided are such methods, wherein the gene SPIKE comprises a polynucleotide sequence selected from the group consisting of: SEQ ID NO: 1; SEQ ID NO: 2; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102.

Also provided are such methods, wherein the gene SPIKE comprises a polynucleotide sequence at least 70%, at least 75%, at least 80%, at least 85%, at least 90%, at least 95%, at least 96%, at least 97%, at least 98%, at least 99%, or 100% identical to a sequence selected from the group consisting of: SEQ ID NO: 1; SEQ ID NO: 2; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102.

Also provided are such methods, wherein the gene SPIKE comprises a polynucleotide sequence at least 70%, at least 75%, at least 80%, at least 85%, at least 90%, at least 95%, at least 96%, at least 97%, at least 98%, at least 99%, or 100% identical to SEQ ID NO: 2.

Also provided are such methods, wherein the gene SPIKE is identified by detecting a first upstream molecular marker selected from the group consisting of: RM5503; RM3423; and Ind4, and a second downstream molecular marker selected from the group consisting of: RM6909; AGT3; RM17487; RM17486; and Ind12.

Also provided are such methods, wherein the gene SPIKE is identified by detecting a first upstream molecular marker selected from the group consisting of: RM3423; and Ind4, and a second downstream molecular marker selected from the group consisting of: AGT3; RM17487; RM17486; and Ind12, wherein the first upstream and second downstream molecular markers are detected using corresponding forward and reverse primers listed in Table 1.

Also provided are such methods, wherein the gene SPIKE is identified by detecting a first molecular marker of about 105 base pairs, Ind2 (forward primer: ACAAGAAGCCGGGAAACCTA (SEQ ID NO: 27); reverse primer: CTCCTCCGGTCCTCCTTAAC (SEQ ID NO: 28)), and a second molecular marker of about 252 base pairs, RM17487 (forward primer: CGGAGCATGTGGAGAGGAACTCG (SEQ ID NO: 55); reverse primer: GGAGAGGGCAAGGGCTTCTTCG (SEQ ID NO: 56)).

The present invention also provides methods of producing an inbred rice plant with improved grain yield comprising: producing a rice plant with improved grain yield according to a method provided herein; crossing the rice plant produced with itself or another rice plant to yield progeny rice seed; growing the progeny rice seed to yield additional rice plants with improved grain yield; and repeating the crossing and growing steps from 0 to 7 times to generate an inbred rice plant with improved grain yield.

Also provided are such methods wherein the step of analyzing the second rice plant for the gene SPIKE further comprises the steps of identifying and selecting rice plants that exhibit improved grain yield.

Also provided are such methods wherein the method further comprises the step of selecting homozygote inbred rice plants.

The present invention also provides methods for producing a rice plant with improved grain yield, the method comprising: providing a first rice plant comprising a gene SPIKE; transferring a nucleic acid encoding gene SPIKE from the first rice plant to a second rice plant; analyzing the second rice plant for the gene SPIKE; identifying and selecting a second rice plant comprising the gene SPIKE and exhibiting improved grain yield when compared to the second rice plant prior to the transfer.

Also provided are such methods, wherein the transfer of the nucleic acid from the first rice plant to the second rice plant is performed by crossing the first rice plant with the second rice plant to produce offspring plants comprising the gene SPIKE, and wherein the steps of analyzing the second rice plant for the gene SPIKE and identifying and selecting a second rice plant comprising the gene SPIKE and exhibiting improved grain yield when compared to the second rice plant prior to the transfer are performed on one or more offspring plants.

Also provided are such methods, wherein the transfer of nucleic acid from the first rice plant to the second rice plant is performed by a transgenic method, by crossing, by backcrossing, by protoplast fusion, by a doubled haploid technique, or by embryo rescue.

Also provided are such methods, wherein backcrossing results in introgression of the gene SPIKE, and recovery of the second rice plant's genome of at least 85%, at least 87%, at least 90%, at least 92%, at least 94%, at least 96%, or at least 98%.

Also provided are such methods, wherein the recovery of the second rice plant's genome is between 92% and 97%.

Also provided are such methods, wherein the step of identifying and selecting a second rice plant comprising the gene SPIKE and exhibiting improved grain yield when compared to the second rice plant prior to the transfer further comprises subjecting the second rice plant to a bioassay for measuring grain yield.

The present invention also provides rice plants with improved grain yield, or part thereof, produced by a method herein, wherein the rice plant or part thereof comprises the gene SPIKE, and wherein the gene SPIKE is not in its natural genetic background.

Also provided are such methods, wherein the first rice plant is selected from an isogenic line of rice plants derived from New Plant Type (NPT) cultivar YP9.

Also provided are such methods, wherein the first rice plant is selected from the Oryza sativa subspecies tropical japonica.

Also provided are such methods, wherein the first rice plant is Daringan.

Also provided are such methods, wherein the second rice plant is selected from the Oryza sativa subspecies indica.

Also provided are such methods, wherein the second rice plant is selected from the group consisting of: PSBRc18; Ciherang; TDK1; BR11; and Swarna.

The present invention also provides transgenic plant cells comprising: at least one plant promoter; and at least one polynucleotide encoding a polypeptide sequence at least 70% identical to that of a protein SPIKE (SEQ ID NO: 3); wherein the promoter and polynucleotide are operably linked and incorporated into the plant cell chromosomal DNA.

Also provided are such methods, wherein the type of cell is selected from the group consisting of: rice; wheat; sorghum; and maize.

Also provided are such methods, wherein the plant cell is homozygous for the gene SPIKE.

The present invention also provides transgenic plants comprising a plurality of cells of a plant herein.

The present invention also provides transgenic plants comprising: at least one plant promoter; and at least one polynucleotide sequence at least 70% identical to that of SPIKE; wherein the promoter and polynucleotide are operably linked and incorporated into the plant chromosomal DNA.

The present invention also provides plants wherein the plant is selected from the group consisting of: rice; wheat; sorghum; and maize.

Also provided are such plants, wherein the plant is a rice plant.

Also provided are such plants, wherein the polynucleotide sequence is at least 70%, at least 75%, at least 80%, at least 85%, at least 90%, at least 95%, at least 96%, at least 97%, at least 98%, at least 99%, or 100% identical to that of SPIKE.

Also provided are such plants, wherein the plant is homozygous for the gene SPIKE.

The present invention also provides seed of a plant herein.

The present invention also provides plant parts of a plant herein.

The present invention also provide plants herein, wherein said plant exhibits a phenotype selected from the group consisting of: increased grain yield per m² relative to a corresponding non-transgenic plant; increased total spikelet number per panicle relative to a corresponding non-transgenic plant; and increased flag leaf width relative to a corresponding non-transgenic plant.

The present invention also provides methods for selecting transgenic plants comprising: screening a population for increased grain yield, wherein plants in the population comprise at least one transgenic plant cell having recombinant DNA incorporated into its chromosomal DNA wherein said recombinant DNA comprises a promoter that is functional in a plant cell and that is functionally linked to an open reading frame of a polynucleotide sequence at least 70% identical to that of SPIKE, wherein individual plants in said population that comprise at least one transgenic plant cell exhibit a grain yield the same as or greater than a grain yield in control plants which do not comprise at least one transgenic plant cell; and selecting from the population one or more plants that exhibit a grain yield greater than the grain yield in control plants which do not comprise at least one transgenic plant cell.

Also provided are such methods which further comprise the step of collecting seeds from the one or more plants selected during the step of electing from the population one or more plants that exhibit a grain yield greater than the grain yield in control plants which do not comprise at least one transgenic plant cell.

Also provided are such methods which further comprise verifying that said recombinant DNA is stably integrated into the selected plant; and analyzing tissue of the selected plant to determine the expression of a polynucleotide sequence at least 70% identical to that of SPIKE.

The present invention also provides methods of increasing grain yield in a cereal grass comprising: crossing a plant of a first variety of a cereal grass, wherein the first variety comprises chromosomal DNA that include a polynucleotide sequence corresponding to gene SPIKE, with a second variety of a cereal grass, wherein the second variety does not comprise chromosomal DNA that includes a polynucleotide sequence corresponding to gene SPIKE; selecting one or more progeny plants having chromosomal DNA that includes the polynucleotide sequence corresponding to gene SPIKE; backcrossing the selected progeny plants to produce backcross progeny plant; selecting one or more backcross progeny plants having chromosomal DNA that includes the polynucleotide sequence corresponding to gene SPIKE; repeating the steps of backcrossing the selected progeny plants to produce backcross progeny plant and selecting one or more backcross progeny plants having chromosomal DNA that includes the polynucleotide sequence corresponding to gene SPIKE one or more times to produce third or higher generation backcross progeny plants having chromosomal DNA that includes the polynucleotide sequence corresponding to gene SPIKE, and all of the physiological and morphological characteristics of the second variety of a cereal grass prior to crossing with the first variety of a cereal grass.

Also provided are such methods, wherein the cereal grass is selected from the group consisting of: rice; wheat; sorghum; and maize.

Also provided are such methods, wherein the cereal grass is rice.

Also provided are such methods, wherein the first variety of a cereal grass is selected from an isogenic line of rice plants derived from New Plant Type (NPT) cultivar YP9.

Also provided are such methods, wherein the first variety of a cereal grass is selected from the Oryza sativa subspecies tropical japonica.

Also provided are such methods, wherein the first variety of a cereal grass is Daringan.

Also provided are such methods, wherein the second variety of a cereal grass is selected from the Oryza sativa subspecies indica.

Also provided are such methods, wherein the second variety of a cereal grass is selected from the group consisting of: PSBRc18; Ciherang; TDK1; BR11; and Swarna.

The present invention also provides methods to cultivate a cereal grass plant, comprising cultivating a seed herein.

The present invention also provides methods to cultivate a cereal grass plant, comprising cultivating a plant part herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D: Characterization of yield-related traits of a near-isogenic line (NIL) for SPIKE. FIG. 1A: Photograph showing plant morphologies of IR64 and NIL-SPIKE. Scale bar: 20 cm. FIG. 1B: Photograph showing panicle structures of IR64 and NIL-SPIKE. Scale bar: 10 cm. FIG. 1C: Photograph showing flag leaves of IR64 (left leaf) and NIL-SPIKE (right leaf). Scale bar: 5 cm. FIG. 1D: Photographs of cross-sections of panicle neck of IR64 and NIL-SPIKE. Scale bar: 500 μm.

FIGS. 1E-1J: Characterization of yield-related traits of a near-isogenic line (NIL) for SPIKE. Bar graphs showing comparisons between IR64 and NIL-SPIKE of (FIG. 1E) spikelet number per panicle (n=8), (FIG. 1F) flag leaf width (n=9), (FIG. 1G) root dry weight at maturity (n=10), (FIG. 1H) rate of chalkiness in brown rice, (FIG. 1I) number of vascular bundles in panicle neck (n=20), and (FIG. 1J) grain weight per m² among two dry (DS) and wet seasons (WS). Percentages above bars in FIG. 1J are yield increases of the NIL relative to IR64. Values are means, with whiskers showing s.d. (s.e.m. in FIG. 1J). ***Significant at 0.1%; **1%; *5%; n.s., not significant.

FIG. 2A: Map-based cloning and expression analysis of SPIKE. A high-resolution map narrowed the SPIKE locus to an 18.0-kbp region between Ind4 and Ind12. The candidate gene is indicated in red. The squares indicates an artifact of gene model prediction. Numbers below the map show the number of recombinants.

FIG. 2B: Map-based cloning and expression analysis of SPIKE. Semi-quantitative expression analysis of SPIKE in culm, leaf, leaf sheath, and root of IR64 and NIL-SPIKE (NIL).

FIGS. 2C-2D: Map-based cloning and expression analysis of SPIKE. Photographs showing production of GUS driven by the NIL-SPIKE promoter in (C) cross-sections of crown roots and lateral roots (scale bar: 50 μm) and (D) young panicles. Scale bar: 2 mm.

FIG. 2E: Map-based cloning and expression analysis of SPIKE. Bar graph showing quantitative expression analysis of SPIKE in 3-5-, 6-10-, 11-20-, and 21-50-mm stages of young panicle of IR64 and NIL-SPIKE. Expression is calibrated to the 3-5-mm panicle stage of IR64. Values are means of three replications, with whiskers showing s.e.m. *Significant at 5%; n.s., not significant.

FIGS. 3A-3D: Transgenic analysis for SPIKE through overexpression and gene silencing. (FIG. 3A) Photograph showing morphologies of IR64 plant and Ubi:SPIKE plant in which SPIKE is overexpressed by the ubiquitin promoter. Scale bar: 20 cm. (FIG. 3B) Photograph showing panicle structures of IR64 and Ubi:SPIKE. Scale bar: 5 cm. Bar graphs showing (FIG. 3C) spikelet number per panicle and (FIG. 3D) flag leaf width of IR64 (n=17) and Ubi:SPIKE plants carrying a single copy (n=20) and five copies (n=13).

FIGS. 3E-3H: Transgenic analysis for SPIKE through overexpression and gene silencing. (FIG. 3E) Photograph showing morphologies of NIL-SPIKE plant and transgenic plant in which SPIKE is silenced by amiRNA. Scale bar: 20 cm. (FIG. 3F) Photograph showing panicle structures of NIL-SPIKE and transgenic plants. Scale bar: 5 cm. Bar graphs showing (FIG. 3F) spikelet number per panicle and (FIG. 3H) flag leaf width of NIL-SPIKE (n=5) and of amiRNA1 (n=4) and amiRNA transgenic plants (n=3). Values are means, with whiskers showing s.d. Results of Tukey-Kramer test for multiple comparisons at the 5% level are shown in C, D, G, and H.

FIGS. 4A-4B: SPIKE increases grain yield in indica genetic background. Gene location (blue ellipses) and photographs showing plant morphology of (FIG. 4A) New Plant Type cultivar YP4 and (FIG. 4B) IRRI146 and IRRI146-SPIKE. Scale bars: 20 cm.

FIGS. 4C-4F: Transgenic analysis for SPIKE through overexpression and gene silencing. Bar graphs showing comparison between IRRI146 and IRRI146-SPIKE of (FIG. 4C) grain weight per m², (FIG. 4D) spikelet number per panicle, and (FIG. 4E) flag leaf width (n=0). (FIG. 4F) Bar graphs showing comparison of spikelet number per panicle between indica cultivars with and without SPIKE PSBRc18 (from Philippines, n=10), TDK1 (from Laos, n=10), Ciherang (from Indonesia, n=13), Swarna (from India, n=17), and BR11 (from Bangladesh, n=27) characterized in the field at IRRI, Philippines. Values are means, with whiskers showing s.e. ***Significant at 0.1%; **1%; *5%.

FIG. 5: Diagram showing breeding scheme for the development of near-isogenic lines for a QTL for total spikelet number per panicle (NIL-SPIKE; right) and of populations segregating at SPIKE. YTH326, with high spikelet number, has introgressed segments from tropical japonica Daringan; YTH326 was selected from BC3 progeny for genetic analysis. NIL-SPIKE was selected by foreground and background selection using DNA markers. The gel pictures show genotypes of SPIKE region by flanking markers RM17483 and RM17486.

FIGS. 6A-6D: Bar graphs showing morphological traits of IR64 (blue) and NIL-SPIKE (orange) in the wet season of 2011 (2011WS) and the dry season of 2012 (2012DS): (FIG. 6A) Rate of filled grain (n=20); (FIG. 6B) panicle number per plant (n=20); (FIG. 6C) 1000-grain weight (n=20); (FIG. 6D) days-to-heading (n=12). Values are means with whiskers showing s.d. **Significant at 1% level; *significant at 5% level; n.s., not significant.

FIG. 7: High-resolution mapping for spikelet number per panicle, secondary branch number, and leaf width. The genotypes of plants with recombination between Ind4 and Ind12 are indicated in white for IR64, in gray for YP9 segments. Hatched boxes indicate the regions which have recombination. Numbers in parentheses show the number of plants which had recombination between molecular markers. Values are means with whiskers showing s.d. **Significant at 1% level; *significant at 5% level.

FIG. 8: RT-PCR of three predicted genes within SPIKE candidate region in IR64 and NIL-SPIKE. Primers were designed for the predicted genes Os04g52479, Os04g52500, and Os04g52504. The molecular markers Ex6.2, Ex7.2 and Ex8.1 were developed for Os04g52479 Os04g52500 and Os04g52504, respectively. UBQ5 was a pair of primes for amplifying ubiquitin as a control.

FIG. 9A: Comparison of SPIKE protein sequences among crop species. Diagram showing phylogenetic tree for SPIKE.

FIG. 9B: Comparison of SPIKE protein sequences among crop species. Alignment showing comparison among rice (IR64 is SEQ ID NO: 6 and NIL-SPIKE is SEQ ID NO: 7), Brachypodium (SEQ ID NO: 90), wheat (SEQ ID NO: 91), sorghum (SEQ ID NO: 92), and maize (SEQ ID NO: 93). The gray regions indicate the trypsin-like serine and cysteine protease domain. The red bars indicate the substitutions between IR64 and YP9. Asterisks indicate complete homology; semicolons indicate substitution of amino acid and spaces indicate complete lack of homology. Integers on the right indicate the cumulative number of amino acid residues in each protein.

FIGS. 10A-10C: Expression of GUS driven by NIL-SPIKE promoter. Photographs showing (FIG. 10A) germinated seeds (scale bar: 2 mm), (FIG. 10B) vascular bundles of culm and panicle neck (scale bar: 500 μm), (FIG. 10C) young leaf (scale bar: 2 mm).

FIG. 11A-11B: Comparison of expression of SPIKE and characterization of T₀ plants (Ubi::SPIKE). (FIG. 11A) Expression of SPIKE in Ubi::SPIKE overexpressor plants. UBQ5 and OsActin1 were a primer set for amplifying ubiquitin and actin as a control. (FIG. 11B) Expression of SPIKE in amiRNA gene-silenced plants.

FIG. 11C-11D: Comparison of expression of SPIKE and characterization of T₀ plants (Ubi::SPIKE). (FIG. 11C) Dot graph showing spikelet number per panicle among T₀ overexpressor plants with copy numbers from zero to seven. (FIG. 11D) Dot graph showing flag leaf width among T₀ overexpressed plants with copy numbers from zero to seven.

FIG. 11E: Comparison of expression of SPIKE and characterization of T₀ plants (Ubi::SPIKE). Number of copies through Southern hybridization on DNA that was digested by BamHI. Blue square indicates Ubi:SPIKE(single) plant, while red square indicated Ubi:SPIKE(multi) plant.

FIGS. 12A-12D: Comparison of agronomic traits between wild type (T65) (Green), nal1 mutant (Fn188) (Red), IR64 (Blue), and NIL-SPIKE (Orange): Bar graphs showing comparison of (FIG. 12A) panicle length, (FIG. 12B) flag leaf length, (FIG. 12C) flag leaf width, (FIG. 12D) total spikelet number per panicle. Whiskers indicate s.d.; n=15. Different letters indicate significant difference at 1% level via Tukey-Kramer test for multiple comparison. T65, Fn188, IR64, and NIL-SPIKE were grown in a field at the Tropical Agricultural Research Front, Japan International Research Center for Agricultural Sciences, Ishigaki, Okinawa, Japan, from August to November 2011. In each plot, a single plant was transplanted per hill at 15 days after sowing at 20 cm between hills and 30 cm between rows. We applied 28 kg ha-1 of P, 28 kg ha-1 of K, and 28 kg ha-1 of N as basal fertilizer and applied same amount at tillering stage.

FIGS. 13A-13B: IAA transport in coleoptiles in IR64 and NIL-SPIKE. (FIG. 13A) Bar graph showing comparison of IAA biosynthesis in 0-3-mm coleoptiles of IR64 and NIL-SPIKE on an agar block. (n=6) (FIG. 13B) Bar graph showing comparison of IAA biosynthesis in 1.5-3-mm coleoptiles of IR64 (blue) and NIL-SPIKE (orange) on an agar block. (n=6)

FIG. 13C: IAA transport in coleoptiles in IR64 and NIL-SPIKE. Diagram showing methods for investigating polar IAA transport using coleoptile section (1.5-3.0 mm) (n=3)

FIG. 13D: IAA transport in coleoptiles in IR64 and NIL-SPIKE. Bar graph showing comparison of polar IAA transport in IR64 (blue) and NIL-SPIKE (orange) coleoptiles. Whiskers show s.d. Surface-sterilized seeds were germinated at 27° C. under red light for 2 days and then in darkness for 1 day. For the IAA biosynthesis assay, six coleoptile sections were excised with a razor blade and placed on a 1.2% agar block (3 mm×15 mm×2 mm) and incubated for the indicated time. For the IAA transport assay, three coleoptile sections (1.5-3.0 mm) were put on an agar block for 30 min to deplete IAA, and then on filter paper containing 3 μM IAA in 10 mM phosphate buffer (pH 6.8) to contact the apical or bottom cut surface for 10 min. Then the coleoptiles were placed on a new agar block. After a given time period, the agar blocks were frozen in liquid N2. IAA was determined by GC-SIM-MS.

FIG. 14: Nal1 sequence comparison.

FIG. 15: Nal1 sequence comparison. Diagram showing CLUSTALW multiple sequence alignment for predicted genes 06 (PG06: putative narrow leaf 1), 07 (PG07: putative Lecithin cholesterol acyltransferase), and 08 (PG08: hypothetical protein). Alignments from top to bottom: Rice_cDNA; EST; Predgeneset; AutoPredgeneset; Genscan_arabi; Genscan_maize; fgenesh_mono; RiceHMM; blastx_nr; mzef; AutoPredLTR; RepeatMasker; tRNAscan; tRNA scan; RepeatMasker; AutoPredLTR; mzef; blastx_nr; RiceHMM; fgenesh_mono; Genscan_maize; Genscan_arabi; AutoPredneneset; Predgeneset; EST; and Rice_cDNA.

FIGS. 16A-16B: Comparison of TSN and FLW among IR64, NIL-SPIKE, and NIL-qTSN4.6. Bar graphs comparing (FIG. 16A) flag leaf width and (FIG. 16B) total spikelet number between IR64, NIL (NIL-SPIKE from YP9), FVW29 (NIL-qTSN4.6 from Nipponbare), FVW 32 (NIL-qTSN4.6 from Nipponbare), and FVW34(NIL-qTSN4.6 from Nipponbare). FLW of NIL-qTSN4.6 is the same as that of NIL-SPIKE, while TSN of NIL-qTSN4.6 is an intermediate phenotype between IR64 and NIL-SPIKE.

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

### DEFINITIONS

“Yield” describes the amount of grain produced by a plant or a group, or crop, of plants. Yield can be measured in several ways, including but not limited to, grain yield per m², t ha⁻¹, and average grain yield per plant.

As used herein a “phenotypic trait” is a distinct variant of an observable characteristic, e.g., grain yield, of a plant that may be inherited by a plant or may be artificially incorporated into a plant by processes such as transfection.

As used herein, “introgression” means the movement of one or more genes, or a group of genes, from one plant variety into the gene complex of another as a result of backcrossing.

As used herein a “transgenic plant cell” means a plant cell that is transformed with stably-integrated, non-natural, recombinant DNA, e.g. by Agrobacterium-mediated transformation or by bombardment using microparticles coated with recombinant DNA or other means. A transgenic plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

“Percent identity” describes the extent to which the sequences of DNA or protein segments are invariant throughout a window of alignment of sequences, for example nucleotide sequences or amino acid sequences. Percent identity is calculated over the aligned length preferably using a local alignment algorithm, such as BLASTp. As used herein, sequences are “aligned” when the alignment produced by BLASTp has a minimal e-value.

As used herein “promoter” means regulatory DNA for initializing transcription. A “promoter that is functional in a plant cell” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that imparts enhanced grain yield. A control plant is used to identify and select a transgenic plant that has enhanced grain yield. A suitable control plant can be a non-transgenic plant of the parental line used to generate a transgenic plant, i.e. devoid of recombinant DNA. A suitable control plant may in some cases be a progeny plant of a hemizygous transgenic plant line that does not contain the recombinant DNA, known as a negative segregant.

The term “quantitative trait locus” or “QTL” refers to a polymorphic genetic locus with at least two alleles that reflect differential expression of a continuously distributed phenotypic trait.

The term “associated with” or “associated” in the context of this disclosure refers to, for example, a nucleic acid and a phenotypic trait, that are in linkage disequilibrium, i.e., the nucleic acid and the trait are found together in progeny plants more often than if the nucleic acid and phenotype segregated independently.

The term “marker” or “molecular marker” or “genetic marker” refers to a genetic locus (a “marker focus”) used as a point of reference when identifying genetically linked loci such as a gene or quantitative trait locus (QTL). The term may also refer to nucleic acid sequences complementary to the genomic sequences, such as nucleic acids used as probes or primers. The primers may be complementary to sequences upstream or downstream of the marker sequences. The term can also refer to amplification products associated with the marker. The term can also refer to alleles associated with the markers. Allelic variation associated with a phenotype allows use of the marker to distinguish germplasm on the basis of the sequence.

The term “crossed” or “cross” in the context of this disclosure means the fusion of gametes via pollination to produce progeny (i.e., cells, seeds or plants). The term encompasses both sexual crosses (the pollination of one plant by another) and selfing (self-pollination, i.e., when the pollen and ovule are from the same plant or from genetically identical plants).

Production of Rice Plants with Improved Grain Yield by Transgenic Methods

According to one particular embodiment, a nucleic acid (preferably DNA) sequence comprising the novel gene SPIKE, or a yield-improving part thereof, may be used for the production of a rice plant with improved grain yield. In this aspect, the embodiment provides for the use of SPIKE or yield-improving parts thereof, for producing a rice plant with improved grain yield, and involves the introduction of a nucleic acid sequence comprising SPIKE in an indica rice cultivar. The nucleic acid sequence may be derived from any suitable donor rice plant. Suitable donor rice plants capable of providing a nucleic acid sequence comprising SPIKE, or yield-improving parts thereof, are the tropical japonica landrace Daringan, the NPT cultivar YP9 (IR68522-10-2-2), which was derived from a cross between indica cultivar Shennung 89-366 and Daringan, tropical japonica Bali Ontjer, and progeny of a cross between NPT IR65564-22-2-3 (from Bali Ontjer) and IRRI146. Other related rice plants that exhibit relatively high grain yield and comprise SPIKE may also be utilized as donor plants.

Once identified in a suitable donor rice plant, the nucleic acid sequence that comprises SPIKE, or a yield-improving part thereof, may be transferred to a suitable recipient plant by any method available. For instance, the said nucleic acid sequence may be transferred by crossing a donor rice plant with a susceptible recipient rice plant (i.e. by introgression), by transformation, by protoplast fusion, by a doubled haploid technique or by embryo rescue, or by any other nucleic acid transfer system, optionally followed by selection of offspring plants comprising SPIKE and exhibiting improved grain yield. For transgenic methods of transfer, a nucleic acid sequence comprising SPIKE, or a yield-improving part thereof, may be isolated from the donor plant by using methods known in the art and the isolated nucleic acid sequence may be transferred to the recipient plant by transgenic methods, for instance by means of a vector, in a gamete, or in any other suitable transfer element, such as a ballistic particle coated with said nucleic acid sequence.

Plant transformation generally involves the construction of an expression vector that will function in plant cells. In certain embodiments, such a vector comprises a nucleic acid sequence that comprises SPIKE, or a yield-improving part thereof, and is under control of or operatively linked to a regulatory element, such as a promoter. The expression vector may contain one or more such operably linked gene/regulatory element combinations, provided that at least one of the genes contained in the combinations is SPIKE. The vector(s) may be in the form of a plasmid, and can be used, alone or in combination with other plasmids, to provide transgenic plants that have improve grain yield, using transformation methods known in the art, such as the Agrobacterium transformation system.

Expression vectors can include at least one marker gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the marker gene). Many commonly used selectable marker genes for plant transformation are known in the art, and include, for example, genes that code for enzymes that metabolically detoxify a selective chemical agent which may be an antibiotic or a herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art.

One method for introducing an expression vector into a plant is based on the natural transformation system of Agrobacterium (see e.g. Horsch et al., 1985). Methods of introducing expression vectors into plant tissue include the direct infection or co-cultivation of plant cells with Agrobacterium tumefaciens. Descriptions of Agrobacterium vectors systems and methods for Agrobacterium-mediated gene transfer are provided by Gruber and Crosby, 1993 and Moloney et al., 1989. See also, U.S. Pat. No. 5,591,616. General descriptions of plant expression vectors and reporter genes and transformation protocols and descriptions of Agrobacterium vector systems and methods for Agrobacterium-mediated gene transfer can be found in Gruber and Crosby, 1993. General methods of culturing plant tissues are provided, for example, by Mild et al., 1993 and by Phillips, et al., 1988. A proper reference handbook for molecular cloning techniques and suitable expression vectors is Sambrook and Russell (2001).

Recombinant DNA constructs useful in transgenic methods are assembled using well known methods, and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit, or signal peptides.

Numerous promoters that are active in plant cells have been described. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus. Promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 and rice actin 2 genes, the maize alcohol dehydrogenase gene intron, the maize heat shock protein 70 gene intron and the maize shrunken 1 gene.

Another method for introducing an expression vector into a plant is based on microprojectile-mediated transformation wherein DNA is carried on the surface of microprojectiles. The expression vector is introduced into plant tissues with a ballistic device that accelerates the microprojectiles to speeds of 300 to 600 m/s which is sufficient to penetrate plant cell walls and membranes (See, Sanford et al., 1987, 1993; Sanford, 1988, 1990; Klein et al., 1988, 1992). Another method for introducing DNA to plants is via the sonication of target cells (see Zhang et al., 1991). Alternatively, liposome or spheroplast fusion has been used to introduce expression vectors into plants (see e.g. Deshayes et al., 1985 and Christou et al., 1987). Direct uptake of DNA into protoplasts using CaCl₂ precipitation, polyvinyl alcohol or poly-L-ornithine has also been reported (see e.g., Hain et al. 1985 and Draper et al., 1982). Electroporation of protoplasts and whole cells and tissues has also been described (D'Halluin et al., 1992 and Laursen et al., 1994).

Following transformation of rice target tissues, expression of the above described selectable marker genes allows for preferential selection of transformed cells, tissues and/or plants, using regeneration and selection methods now well known in the art. The markers described herein may also be used for that purpose.

Production of Rice Plants with Improve Grain Yield by Non-Transgenic Methods

In another embodiment for producing a rice plant with improved yield, protoplast fusion can be used for the transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained with plant species that cannot be interbred in nature, is tissue cultured into a hybrid plant exhibiting the desirable combination of traits. More specifically, a first protoplast can be obtained from a rice plant or other plant line that exhibits improved grain yield. For example, a protoplast from Darigan, YP9, or Bali Ontjer can be used. A second protoplast can be obtained from rice or other plant variety, preferably a popular indica rice cultivar. Additionally, the second protoplast may be from a rice variety that comprises commercially desirable characteristics, such as, but not limited to disease resistance, insect resistance, weed resistance, etc. The protoplasts are then fused using traditional protoplast fusion procedures, which are known in the art.

Alternatively, embryo rescue may be employed in the transfer of a nucleic acid comprising SPIKE from a donor plant to a recipient plant. Embryo rescue can be used as a procedure to isolate embryos from crosses wherein plants fail to produce viable seed. In this process, the fertilized ovary or immature seed of a plant is tissue cultured to create new plants.

Traditional breeding techniques can also be used to introgress a nucleic acid sequence encoding SPIKE into a target recipient rice plant in which a higher grain yield is desirable, preferably an indica rice cultivar. In one method, which is referred to as pedigree breeding, a donor rice plant comprising a nucleic acid sequence encoding SPIKE is crossed with a rice plant in which a higher grain yield is desirable, preferably an indica rice cultivar. The resulting plant population (representing the F1 hybrids) is then self-pollinated and set seeds (F2 seeds). The F2 plants grown from the F2 seeds are then screened for improved grain yield. The population can be screened for improved grain yield in a number of different ways. For example, the population can be screened by field evaluation over several seasons. Yield may be determined by grain yield per m² (GYS), weight of grain per hectare (e.g., t ha⁻¹, kg ha⁻¹), average grain weight per plant, or any other method known in the art.

A Rice Plant Having Improved Grain Yield, or a Part Thereof, Obtainable by a Method Described Herein is Also an Embodiment of the Present Invention

One particular embodiment relates to a rice plant having improved grain yield, or part thereof, comprising within its genome SPIKE, or a yield-improving part thereof, wherein SPIKE or the yield improving part thereof is not in its natural genetic background. The rice plants having improved grain yield described herein can be of any genetic type such as inbred, hybrid, haploid, dihaploid, parthenocarp or transgenic. Further, the plants of the present invention may be heterozygous or homozygous for the improved grain yield trait, preferably homozygous. Although SPIKE and yield-improving parts thereof may be transferred to any plant in order to provide for a plant having improved grain yield, the methods and plants described herein are preferably related to the cereal grass family, more preferably rice.

Inbred rice lines having improved grain yield can be developed using the techniques of recurrent selection and backcrossing, selfing and/or dihaploids or any other technique used to make parental lines. In a method of selection and backcrossing, improved grain yield can be introgressed into a target recipient plant (which is called the recurrent parent) by crossing the recurrent parent with a first donor plant (which is different from the recurrent parent and referred to herein as the “non-recurrent parent”). The recurrent parent is a plant in which an increase in grain yield is desirable, preferably an indica rice cultivar. Optionally, the recurrent parent possesses commercially desirable characteristics, such as, but not limited to disease resistance, insect resistance, weed resistance, etc. The non-recurrent parent comprises a nucleic acid sequence that encodes SPIKE. The non-recurrent parent can be any plant variety or inbred line that is cross-fertile with the recurrent parent. The progeny resulting from a cross between the recurrent parent and non-recurrent parent are backcrossed to the recurrent parent. The resulting plant population is then screened. The population can be screened in a number of different ways. F1 hybrid plants that exhibit improved grain yield and comprise the requisite nucleic acid sequence encoding for SPIKE are then selected and selfed and selected for over a number of generations in order to allow for the rice plant to become increasingly inbred. This process of continued selfing and selection can be performed for zero to five or more generations. The result of such breeding and selection is the production of lines that are genetically homogenous for the genes associated with improved grain yield as well as other genes associated with traits of commercial interest.

Instead of using phenotypic pathology screens of bioassays, marker assisted selection (MAS) can be performed using one or more of the herein described molecular markers, hybridization probes, or polynucleotides to identify those progeny that comprise a nucleic acid sequence encoding for SPIKE. Alternatively, MAS can be used to confirm the results obtained from the quantitative bioassays. Once the appropriate selections are made, the process is repeated. The process of backcrossing to the recurrent parent and selecting for improved grain yield is repeated for approximately five or more generations. The progeny resulting from this process are heterozygous for SPIKE. The last backcross generation is then selfed in order to provide for homozygous pure breeding progeny for improved grain yield.

The rice lines having improved grain yield described herein can be used in additional crossings to create hybrid plants having improved grain yield. For example, a first inbred rice plant having improved grain yield produced by methods described herein can be crossed with a second inbred rice plant possessing commercially desirable traits such as, but not limited to, disease resistance, insect resistance, weed resistance, etc. This second inbred rice line may or may not have relatively improved grain yield.

Marker Assisted Selection and Backcrossing

SPIKE marker assisted selection (MAS) and marker assisted back crossing (MABC) are described herein.

Molecular markers can include restriction fragment length polymorphisms (RFLP), random amplified polymorphic DNA (RAPD), amplified fragment length polymorphisms (AFLP), single nucleotide polymorphisms (SNP) or simple sequence repeats (SSR). A primary motivation for development of molecular markers in crop species is the potential for increased efficiency in plant breeding through marker assisted selection (MAS) and marker assisted backcrossing (MABC). Genetic marker alleles are used to identify plants that contain a desired genotype at one or more loci and that are expected to transfer the desired genotype, along with a desired phenotype to their progeny. Genetic marker alleles can be used to identify plants that contain a desired genotype at one locus or at several unlinked or linked loci (e.g., a haplotype) and that would be expected to transfer the desired genotype, along with a desired phenotype to their progeny.

After a desired phenotype, e.g., improved grain yield and a polymorphic chromosomal locus are determined to segregate together, it is possible to use those polymorphic loci to select for alleles corresponding to the desired phenotype: a process called marker-assisted selection (MAS). In brief, a nucleic acid corresponding to the marker nucleic acid is detected in a biological sample from a plant to be selected. This detection can take the form of hybridization of a probe nucleic acid to a marker, e.g., using allele-specific hybridization, Southern analysis, northern analysis, in situ hybridization, hybridization of primers followed by PCR amplification of a region of the marker, or the like. A variety of procedures for detecting markers are described herein. After the presence (or absence) of a particular marker and/or marker allele in the biological sample is verified, the plant is selected, i.e., used to make progeny plants by selective breeding.

Screening a large number of plants for improved grain yield can be expensive, time consuming and unreliable. Use of the genetically-linked nucleic acids described herein as genetic markers for improved grain yield is an effective method for selecting plants capable of fertility restoration in breeding programs. For example, one advantage of marker-assisted selection over field evaluations for improved grain yield is that MAS can be done at any time of year regardless of the growing season. Moreover, environmental effects are irrelevant to MAS.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent into an otherwise desirable genetic background from the recurrent parent. The more cycles of backcrossing that are done, the greater the genetic contribution of the recurrent parent to the resulting variety. This is often necessary, because donor parent plants may be otherwise undesirable. In contrast, varieties which are the result of intensive breeding programs may merely being deficient in one desired trait such as improved grain yield. Backcrossing can be done to select for or against a trait.

Markers corresponding to genetic polymorphisms between members of a population can be detected by numerous methods, well-established in the art (e.g., restriction fragment length polymorphisms, isozyme markers, allele specific hybridization (ASH), amplified variable sequences of the plant genome, self-sustained sequence replication, simple sequence repeat (SSR), single nucleotide polymorphism (SNP) or amplified fragment length polymorphisms (AFLP)).

The majority of genetic markers rely on one or more properties of nucleic acids for their detection. For example, some techniques for detecting genetic markers utilize hybridization of a probe nucleic acid to nucleic acids corresponding to the genetic marker. Hybridization formats include but are not limited to, solution phase, solid phase, mixed phase or in situ hybridization assays. Markers which are restriction fragment length polymorphisms (RFLP), are detected by hybridizing a probe (which is typically a sub-fragment or a synthetic oligonucleotide corresponding to a sub-fragment of the nucleic acid to be detected) to restriction digested genomic DNA. The restriction enzyme is selected to provide restriction fragments of at least two alternative (or polymorphic) lengths in different individuals and will often vary from line to line. Determining a (one or more) restriction enzyme that produces informative fragments for each cross is a simple procedure, well known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing. Nucleic acid probes to the marker loci can be cloned and/or synthesized. Detectable labels suitable for use with nucleic acid probes include any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling markers is readily achieved such as by the use of labeled PCR primers to marker loci.

The hybridized probe is then detected using, most typically, autoradiography or other similar detection technique (e.g., fluorography, liquid scintillation counter, etc.). Examples of specific hybridization protocols are widely available in the art.

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are well known in the art. Examples of techniques include in vitro methods, including the polymerase chain reaction (PCR), the ligase chain reaction (LCR), O,β-replicase amplification and other RNA polymerase mediated techniques (e.g., NASBA). Essentially any RNA can be converted into a double stranded DNA suitable for restriction digestion, PCR expansion and sequencing using reverse transcriptase and a polymerase.

Oligonucleotides for use as primers, e.g., in amplification reactions and for use as nucleic acid sequence probes, are typically synthesized chemically according to the solid phase phosphoramidite triester method, or can simply be ordered commercially.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) Rnase H and (3) a DNA-dependent RNA polymerase. By mimicking the retroviral strategy of RNA replication by means of cDNA intermediates, this reaction accumulates cDNA and RNA copies of the original target.

There are many different types of molecular markers, including amplified fragment length polymorphisms (AFLP), allele-specific hybridization (ASH), single nucleotide polymorphisms (SNP), simple sequence repeats (SSR) and isozyme markers. SSR data is generated by hybridizing primers to conserved regions of the plant genome which flank the SSR sequence. PCR is then used to amplify the repeats between the primers. The amplified sequences are then electrophoresed to determine the size and therefore the di-, tri and tetra nucleotide repeats.

The presence of SPIKE in the genome of a plant exhibiting a preferred phenotypic trait is determined by any method listed above, e.g., RFLP, AFLP, SSR, etc. If the nucleic acids from the plant are positive for a desired genetic marker, the plant can be selfed to create a true breeding line with the same genotype or it can be crossed with a plant with the same marker or with other desired characteristics to create a sexually crossed hybrid generation.

The materials and methods of the present invention may be similarly used to confer improved grain yield in cereal grasses other than rice, such as wheat, sorghum, and maize.

### EXAMPLES

The methods and embodiments described herein are further defined in the following Examples, in which all parts and percentages are by weight and degrees are Celsius, unless otherwise stated. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

Results

Characterization of NIL-SPIKE

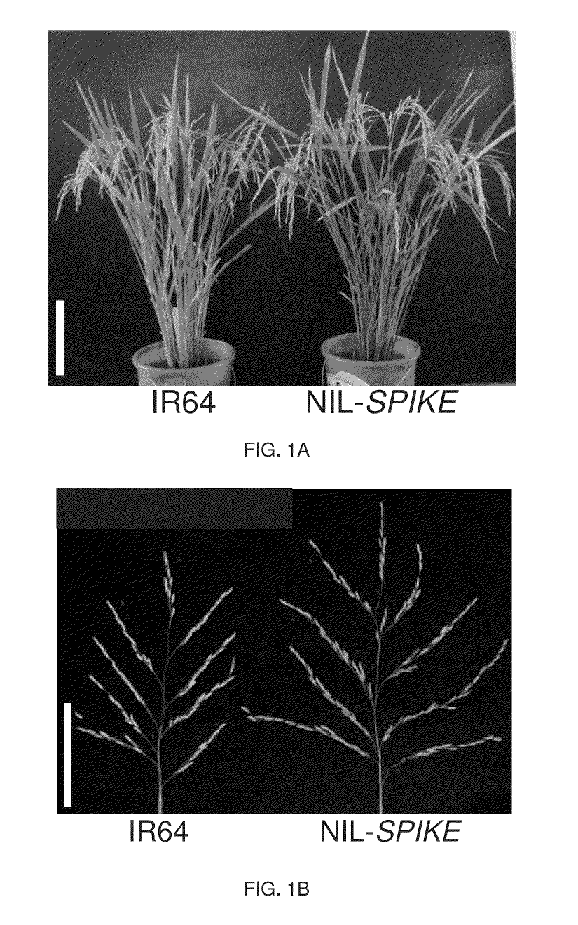

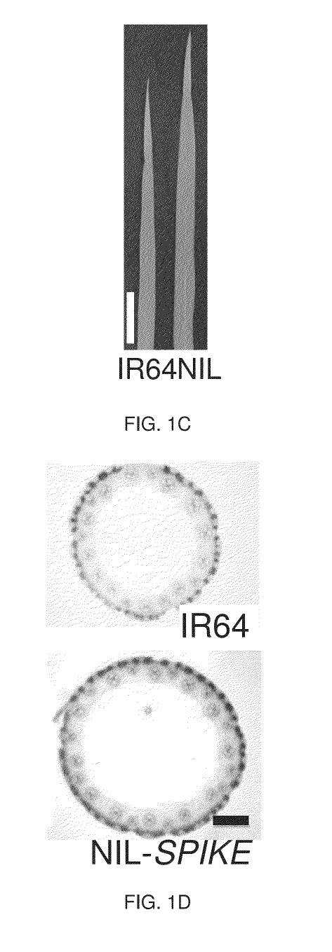

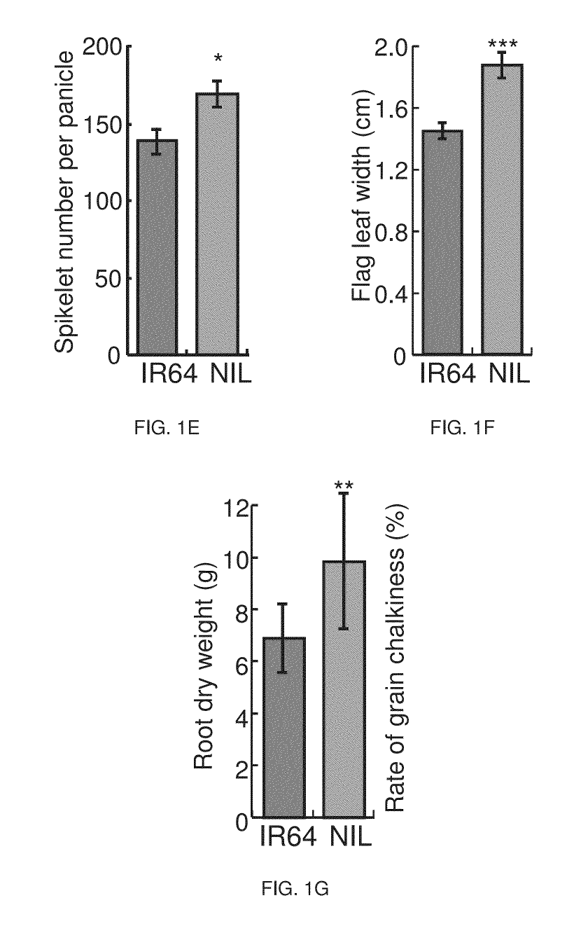

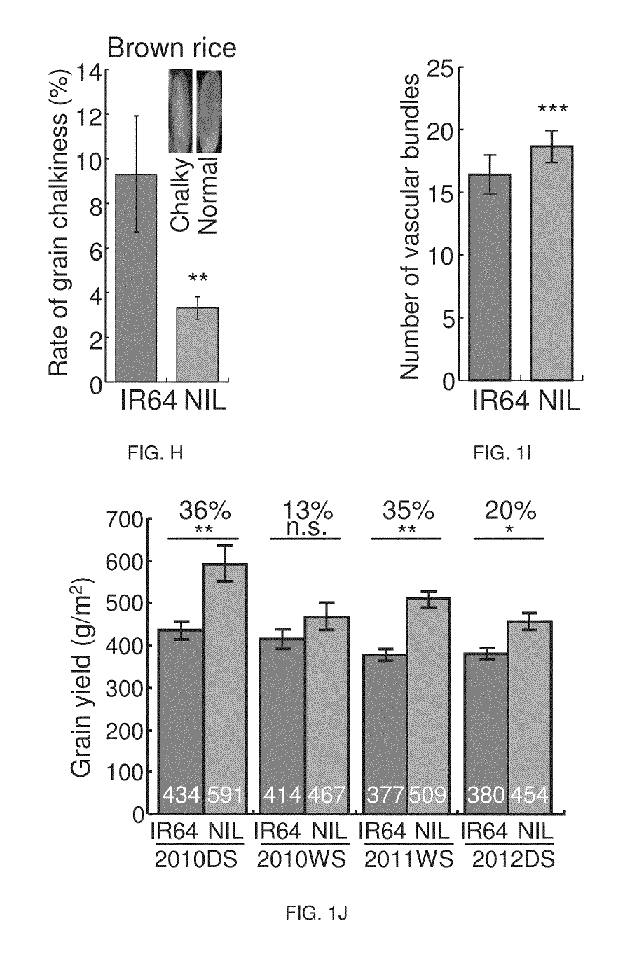

The Quantitative trait locus (QTL) qTSN4, designated here as SPIKE (SPIKELET NUMBER), was characterized by using an NIL for SPIKE, NIL-SPIKE (FIG. 1A). NIL-SPIKE had larger panicles (FIG. 1B), leaves (FIG. 1C), and panicle necks than IR64 (FIG. 1D). Among yield-related traits, it had higher TSN (FIG. 1E), flag leaf width (FLW; FIG. 1F), root dry weight (RDW; FIG. 1G), and rate of filled grain (Fig. S2A), but had lower panicle number per plant and 1000-grain weight (Fig. S2B, C). Notably, along with the rate of filled grain, the grain appearance was improved (FIG. 1H), presumably owing to a strengthening of assimilate supply to the larger number of spikelets by an increase in vascular bundle number (VBN; FIG. 1I). Consequently, the grain yield per m² (GYS) of the NIL was consistently higher than that of IR64 over four cropping seasons, significantly so in three of the four seasons (FIG. 1J). The average GYS of the NIL was 28% higher in the dry season and 24% higher in the wet season than that of IR64 (˜400 g/m²). Therefore, the increase in GYS in the NIL without a decline in grain appearance was achieved through the enlargement of sink size (high TSN), source size (broad FLW and high RDW), and translocation capacity (high VBN). Additionally, days-to-heading was unchanged (Fig. S2D). Thus, SPIKE is highly useful for improving yield without changing locally adapted traits.

High-Resolution Linkage Mapping and Identification of SPIKE

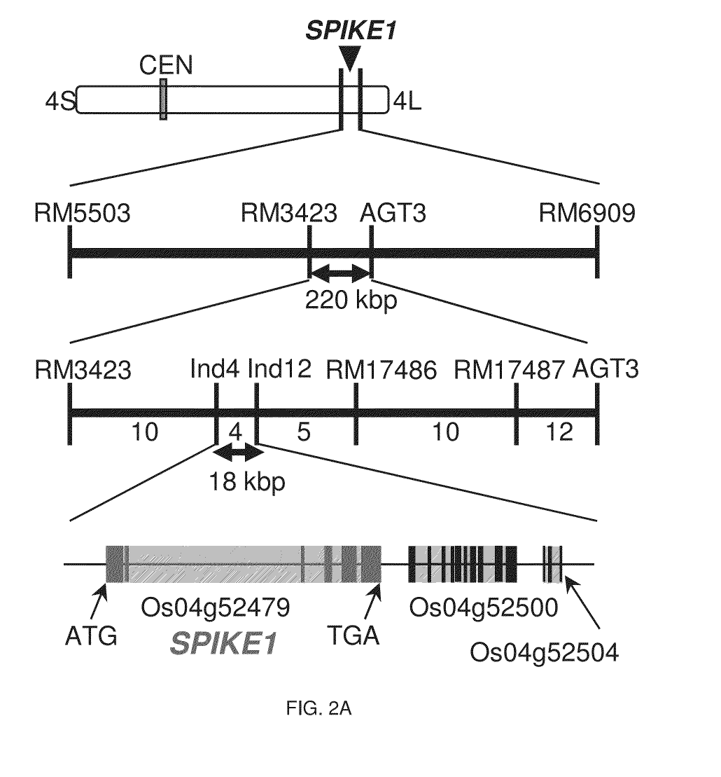

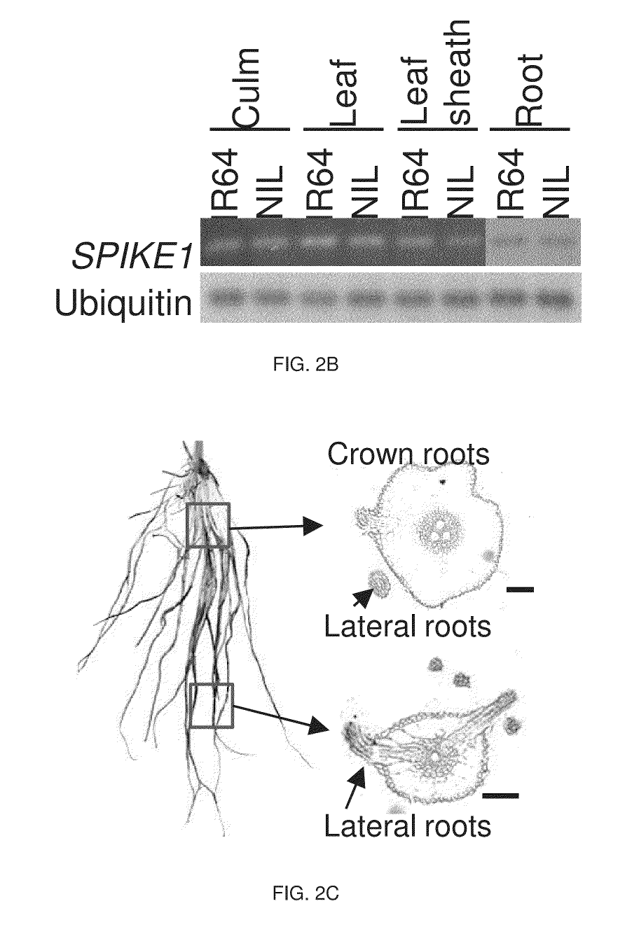

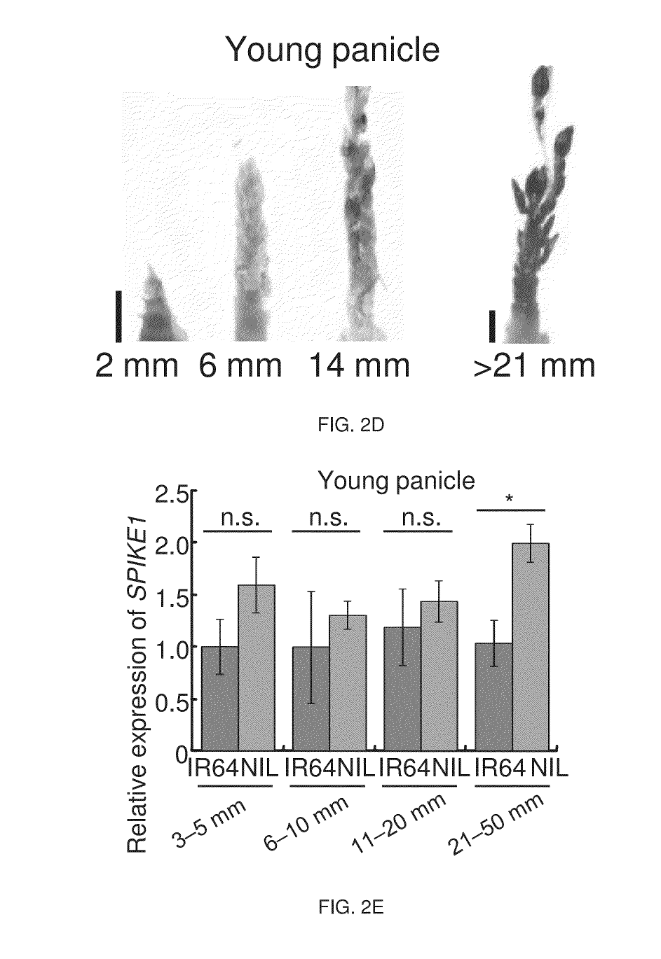

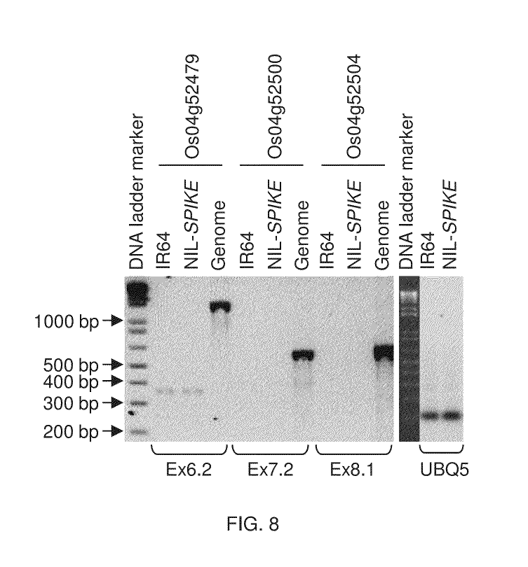

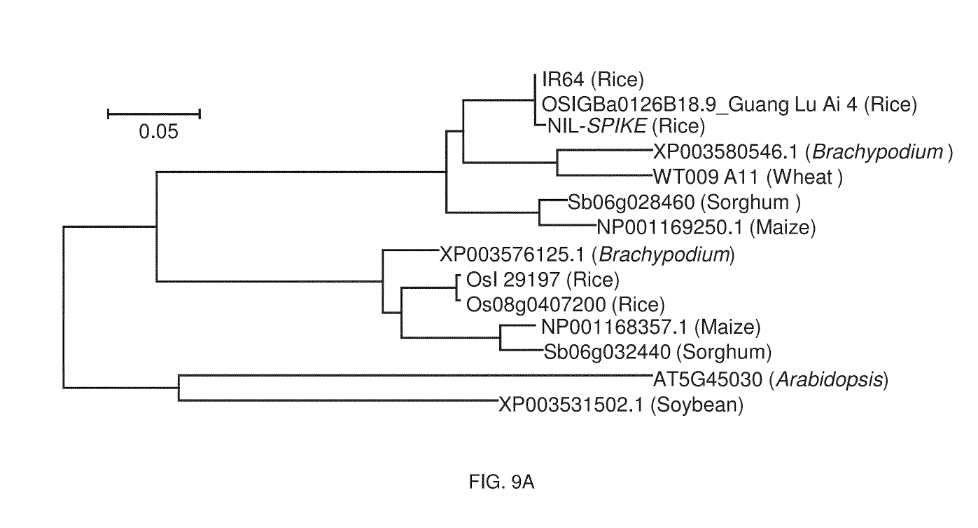

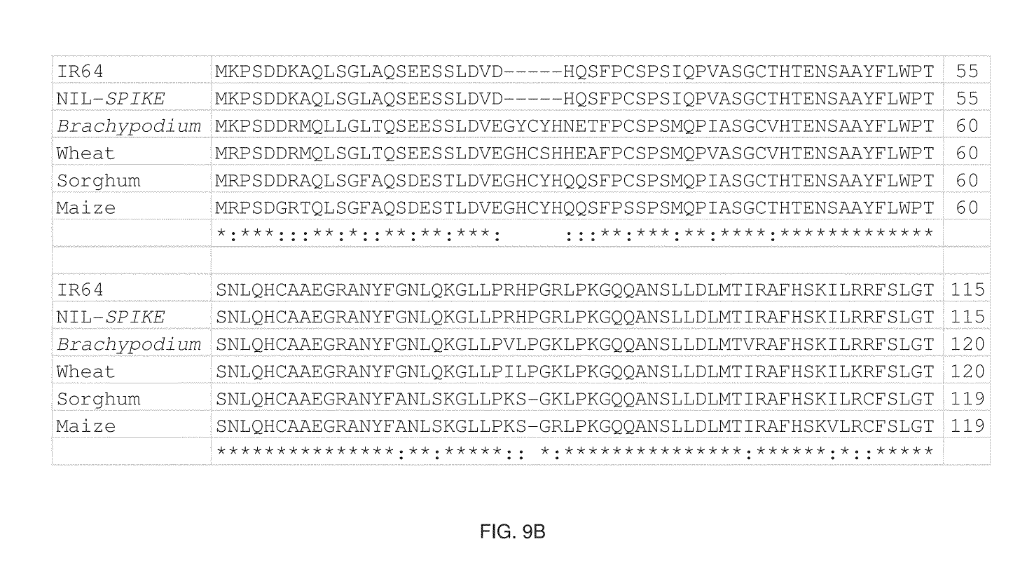

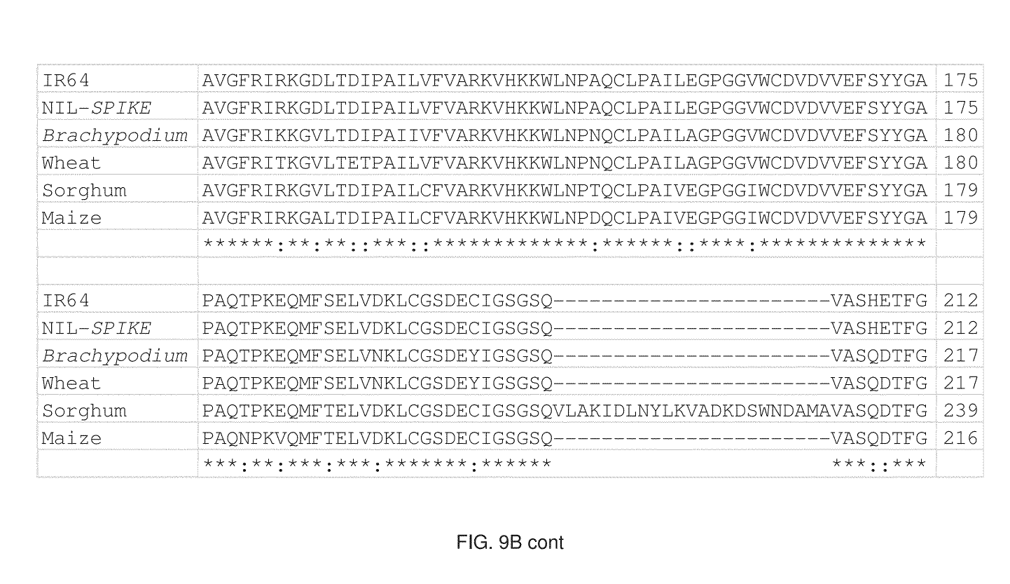

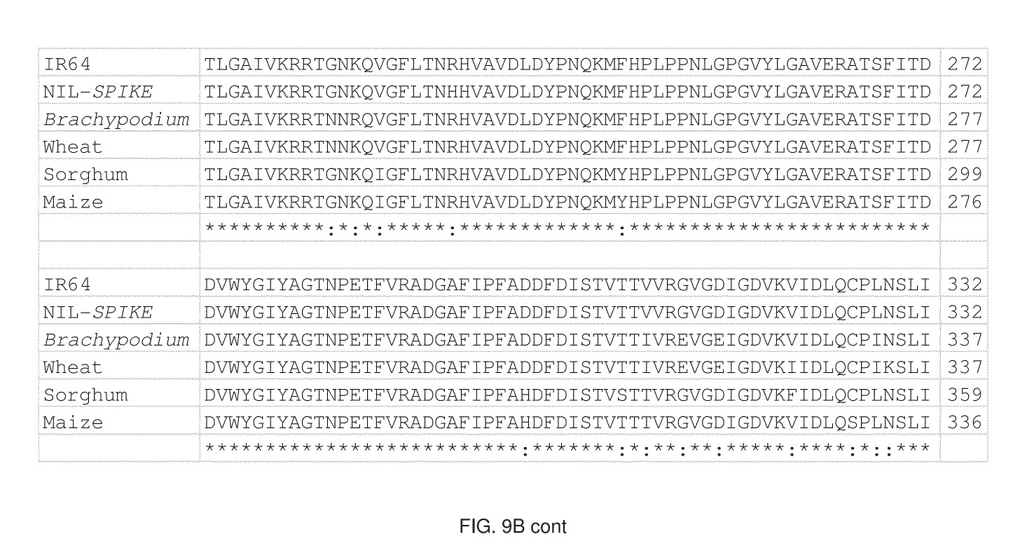

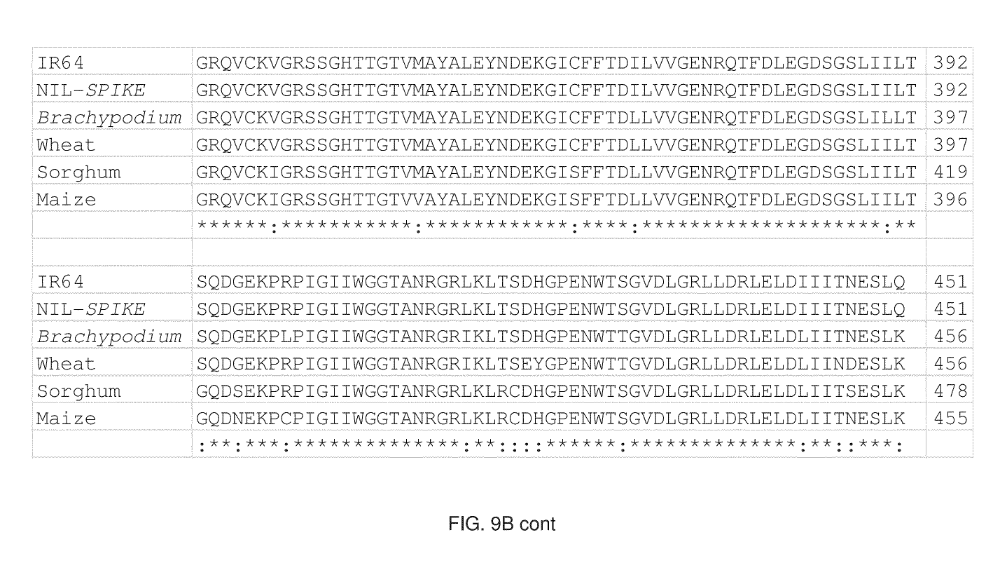

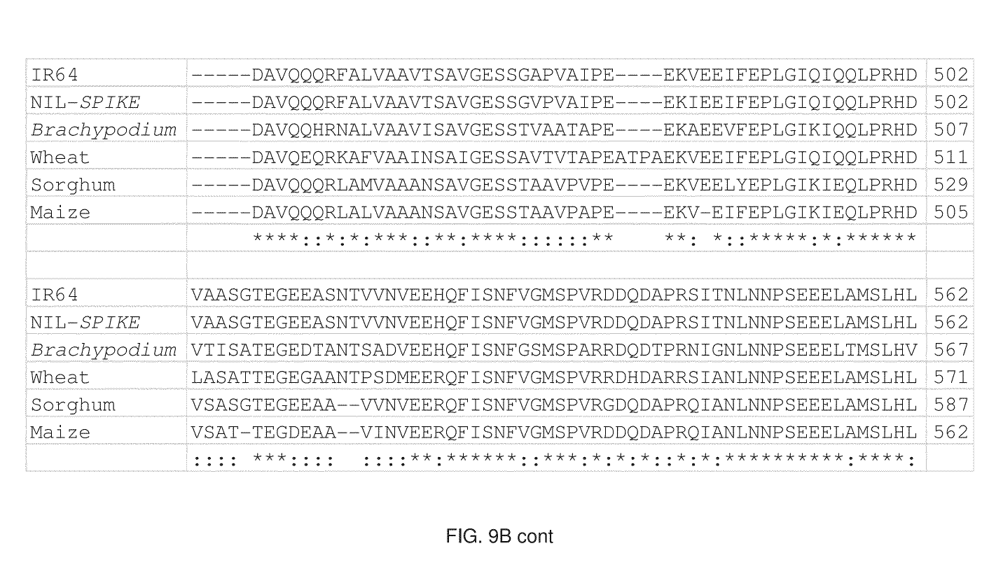

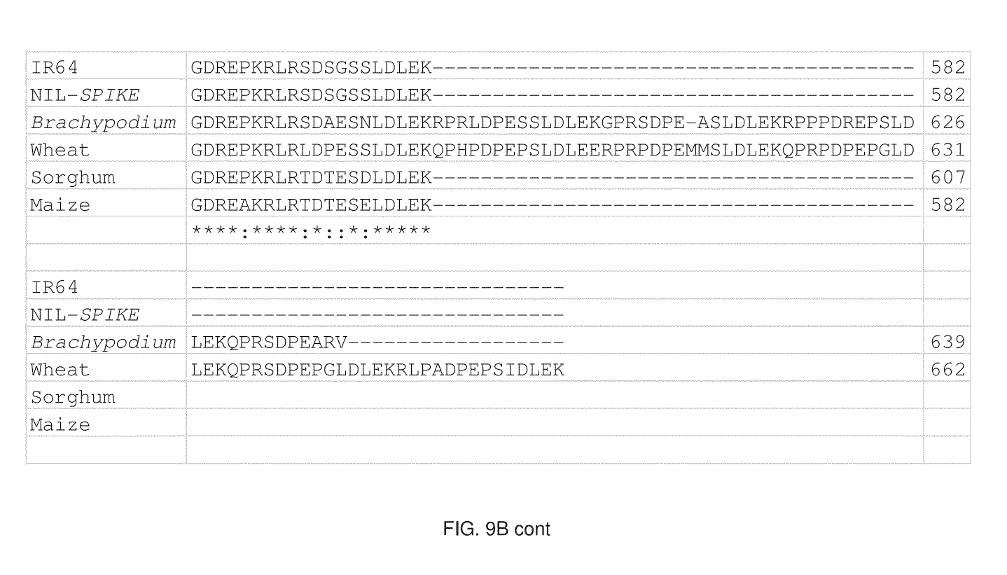

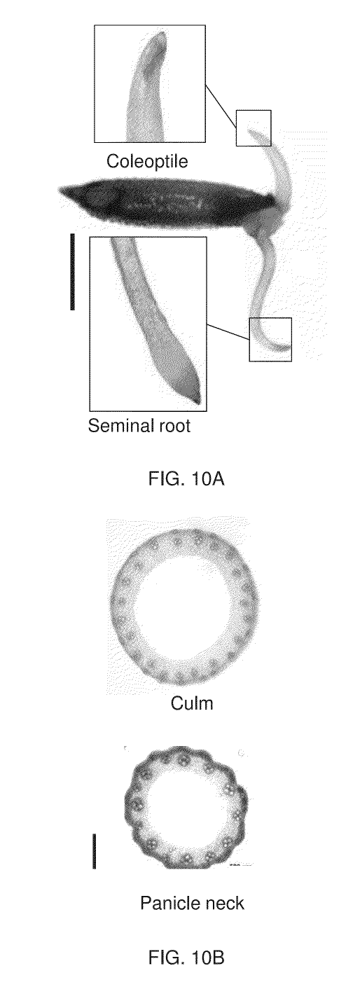

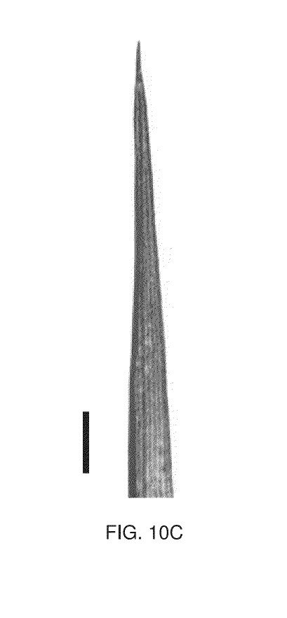

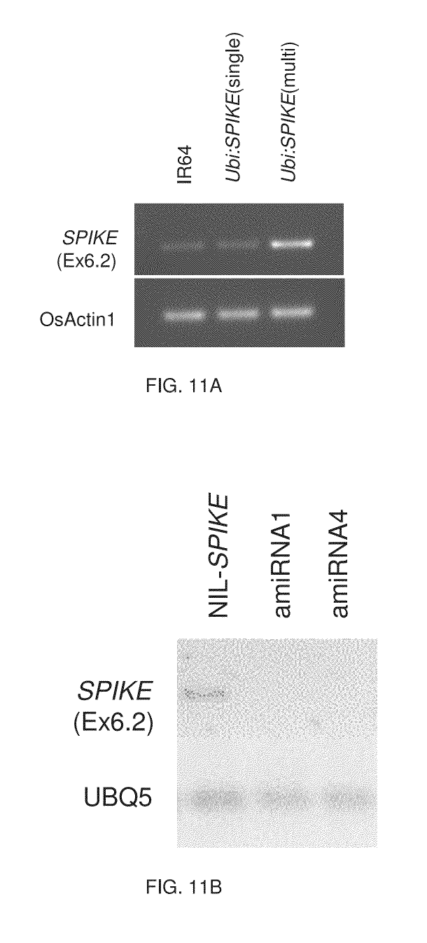

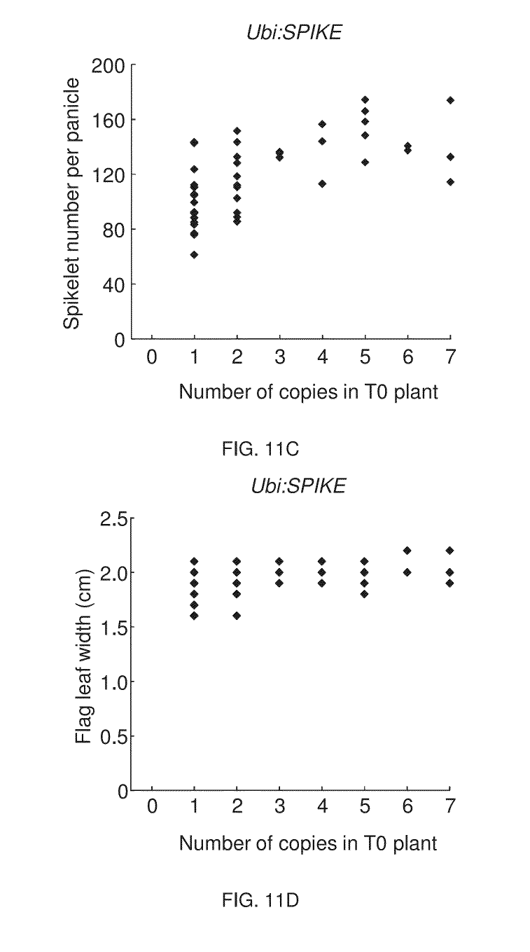

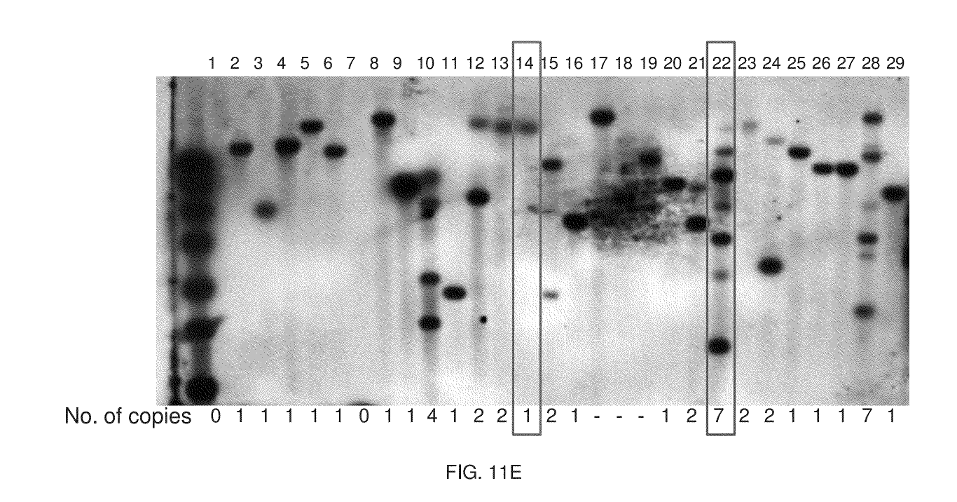

To identify a gene for SPIKE, high-resolution linkage analysis was conducted using 7996 BC₄F₃ plants evaluated for TSN. The candidate region lay between markers Ind4 and Ind12 (18.0 kbp), in which the Rice Genome Annotation Project database at Michigan State University predicts three genes (FIG. 2A). In addition to TSN, the suggested gene was associated with an increase in secondary branch number and leaf width (FIG. 7). Expression analysis in a young panicle revealed that only Os04g52479 (Nal1: NARROW LEAF 1,) was expressed (FIG. 8), and thus is the most probable candidate for SPIKE. Analysis of the predicted amino acid sequence of SPIKE revealed three amino acid substitutions between IR64 and NIL-SPIKE, one of them in the trypsin-like serine and cysteine protease domain (FIG. 9). Further, the SPIKE protein shows >84% identity with proteins of Brachypodium, wheat, sorghum, and maize, and high similarity in the trypsin-like serine and cysteine protease domain. This similarity demonstrates conservation of the biochemical function of the SPIKE protein family among these species.

Expression Analysis of SPIKE

SPIKE was consistently expressed in several organs (FIG. 2B). To analyze the expression of SPIKE during plant development, the β-glucuronidase (GUS) reporter gene was expressed under the control of the native SPIKE promoter in transgenic IR64 plants. Histochemical analysis revealed GUS activity in the coleoptile, vascular bundle at the panicle neck and culm, leaves (FIG. 56A-C), crown roots, lateral roots (FIG. 2C), and young panicles (FIG. 2D). Aside from the coleoptile, the pattern of GUS expression coincided with the organs enlarged in NIL-SPIKE. Quantitative RT-PCR revealed that the expression of SPIKE in young panicles at various stages was consistently higher in NIL-SPIKE than in IR64, and double that of IR64 at the 21-50-mm stage (P=0.05; FIG. 2E). The results show that the increase in SPIKE expression at the young panicle stage increased spikelet number.

Gene Validation of SPIKE Through Transgenic Analysis

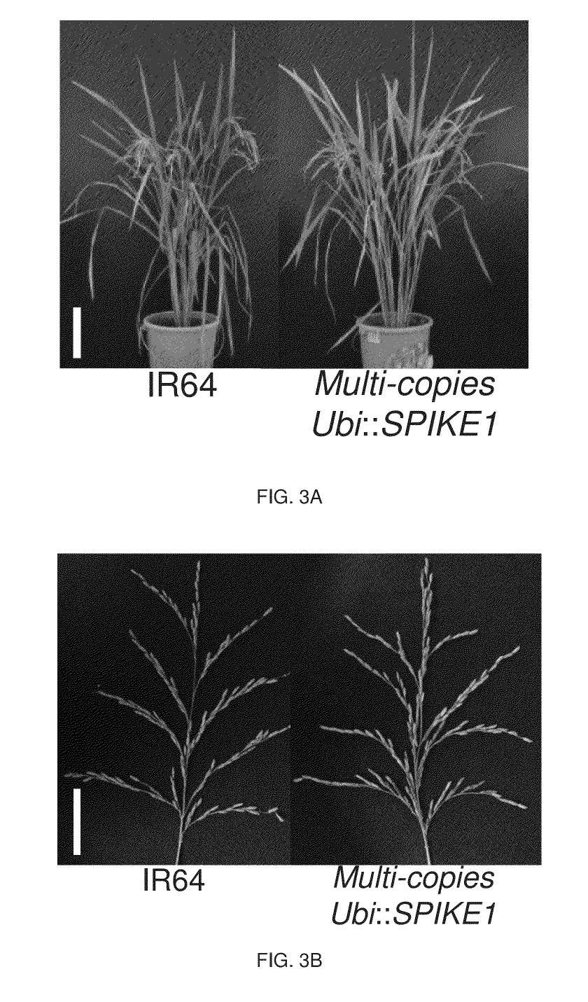

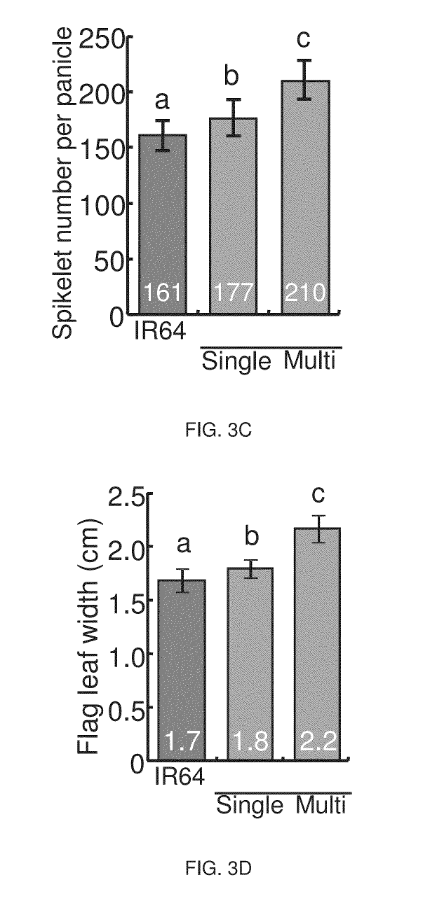

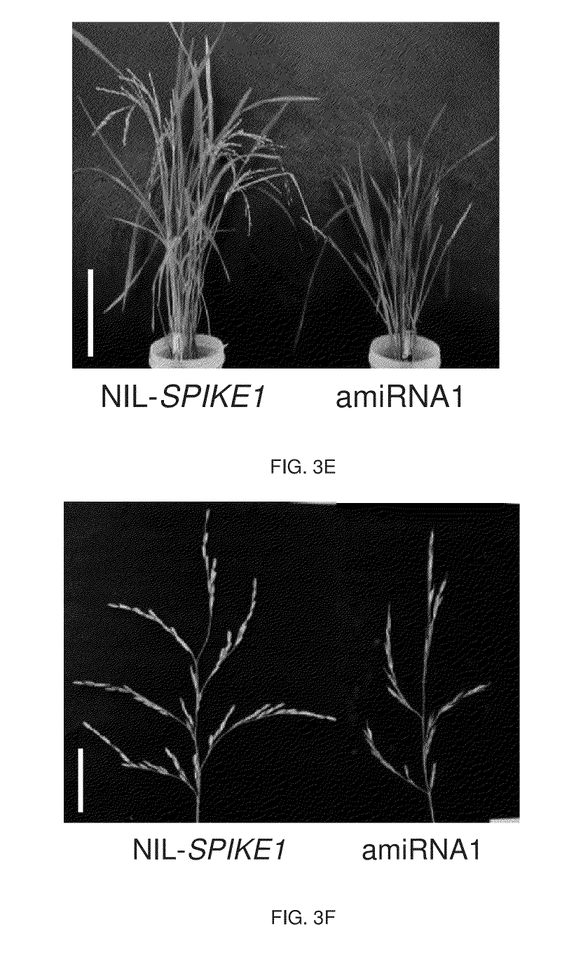

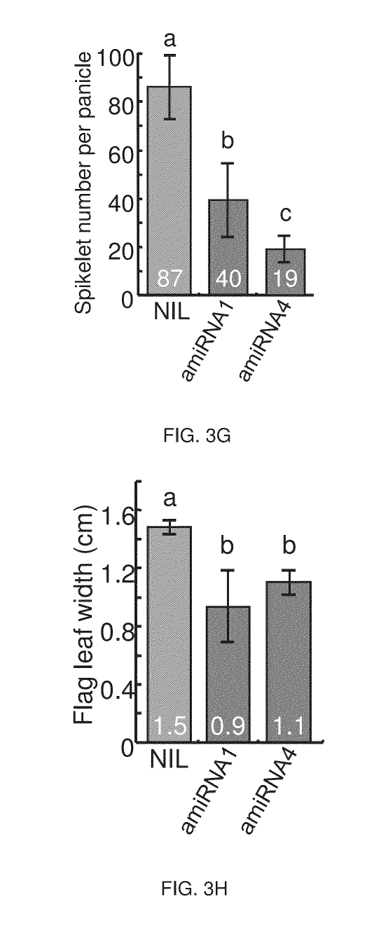

To validate collected data and to gain insight into the function of SPIKE, overexpressor lines (using a constitutive promoter) and silencing lines (using artificial microRNA: amiRNA) were generated. A DNA fragment containing the cDNA of SPIKE from NIL-SPIKE fused with the ubiquitin promoter (Ubi:SPIKE) was introduced into IR64 by transformation. The overexpressor transgenic plants showed a similar phenotype to NIL-SPIKE, including large panicles and broad flag leaves (FIGS. 3A, B). Plants carrying a single copy had significantly greater TSN and FLW than IR64 (FIG. 3C, D). Plants carrying multiple copies had significantly greater TSN and FLW than those with a single copy, suggesting increasing TSN and FLW along with expression of SPIKE. A significant higher transcript in the event carrying multiple copies was observed (Fig. S7A). This suggests the dosage effect of SPIKE transcript on the plant phenotype. Additionally, TSN and FLW of T₀ Ubi:SPIKE plants increased with copy number (FIG. 57C-E). In contrast, transformation of two amiRNA precursors that targeted the first (amiRNA1) and fourth exons (amiRNA4) of SPIKE into NIL-SPIKE to downregulate SPIKE (FIGS. 3E, F; S7B) produced transgenic plants with significantly lower TSN and narrower leaves than NIL-SPIKE (FIG. 3G, H).

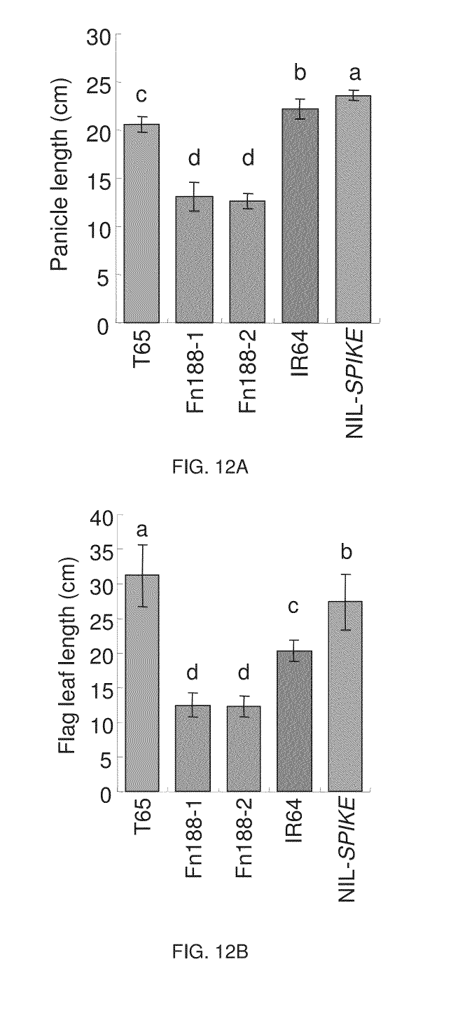

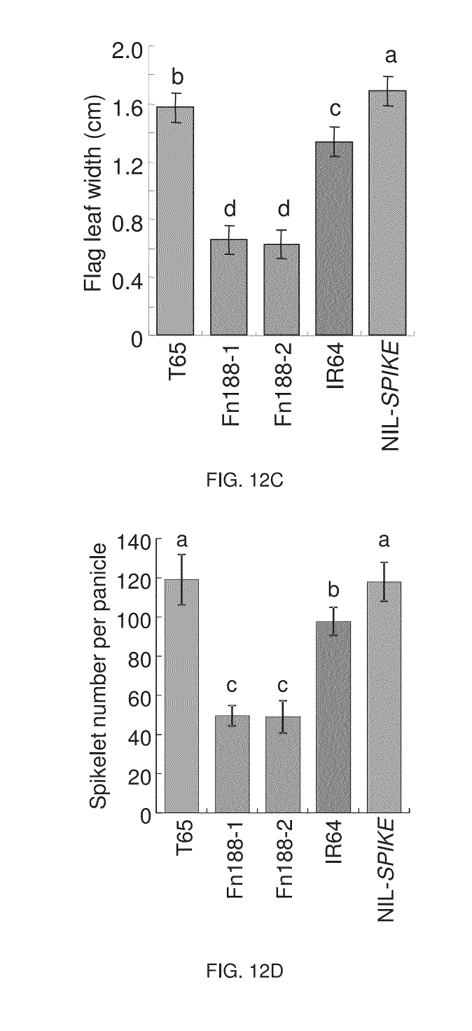

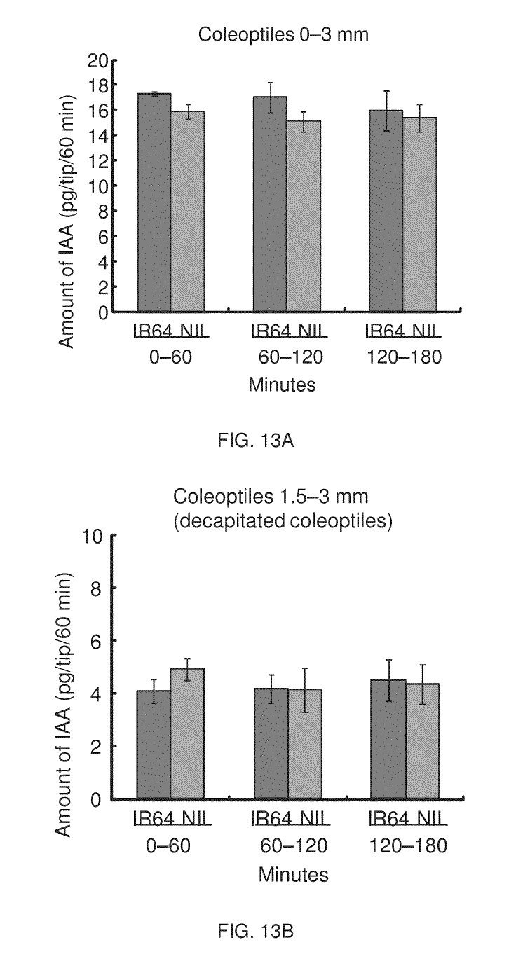

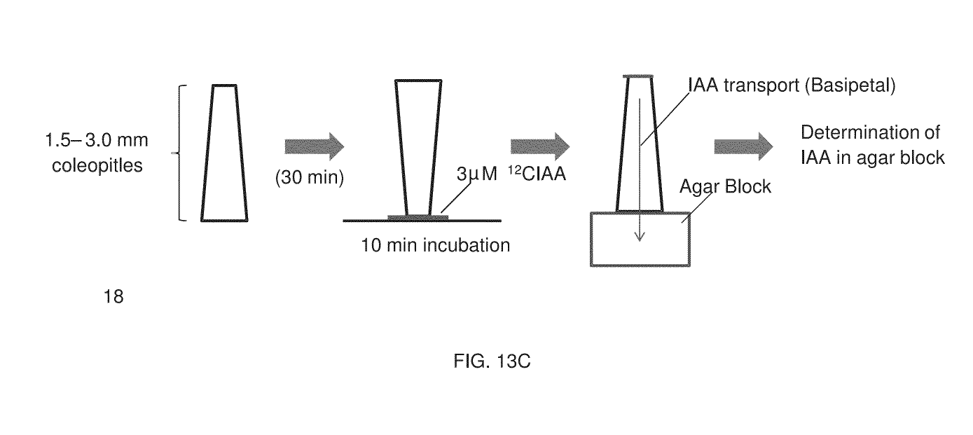

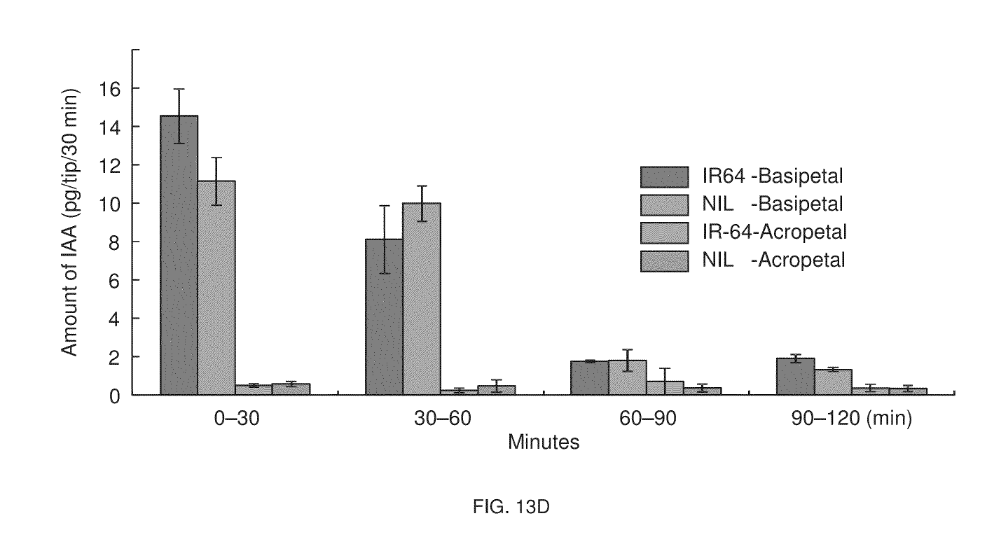

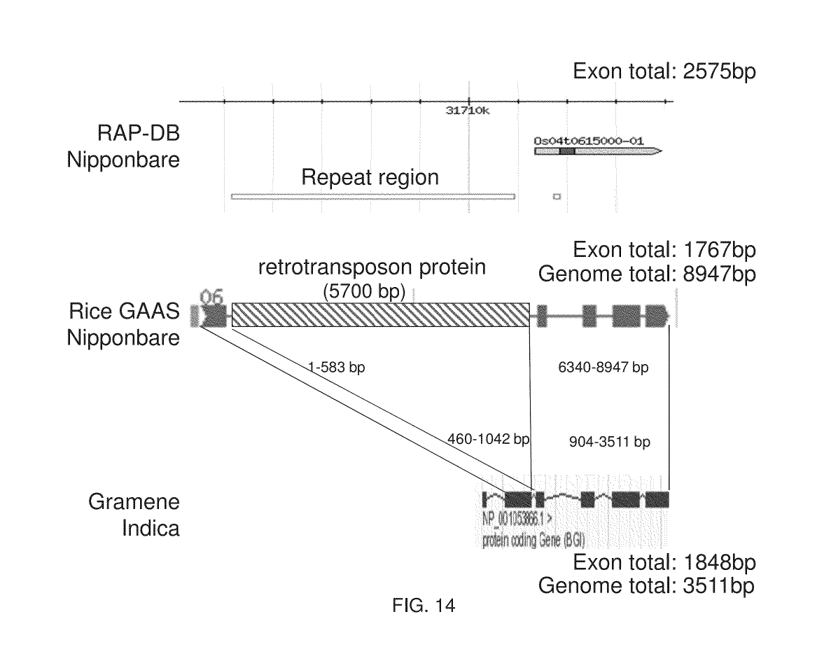

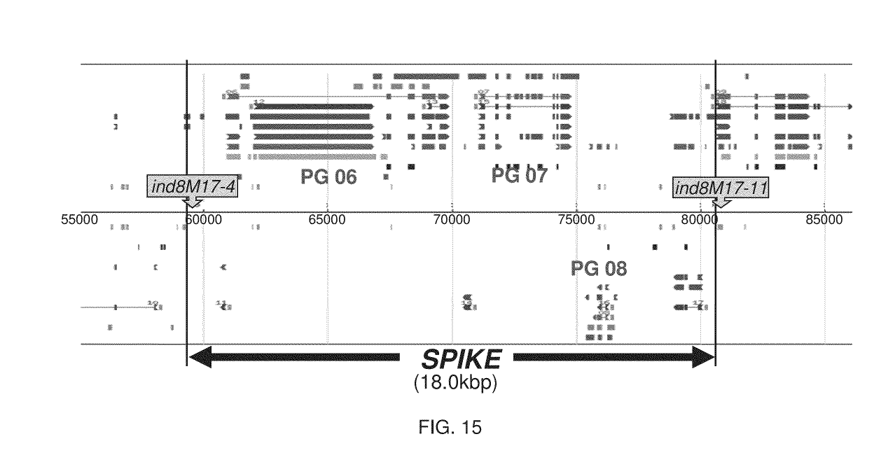

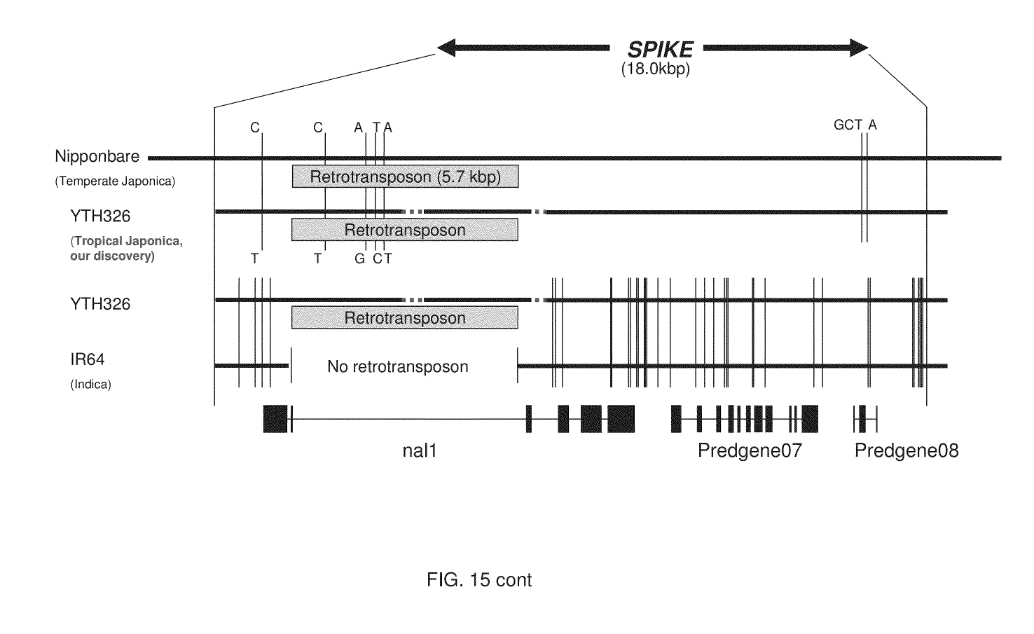

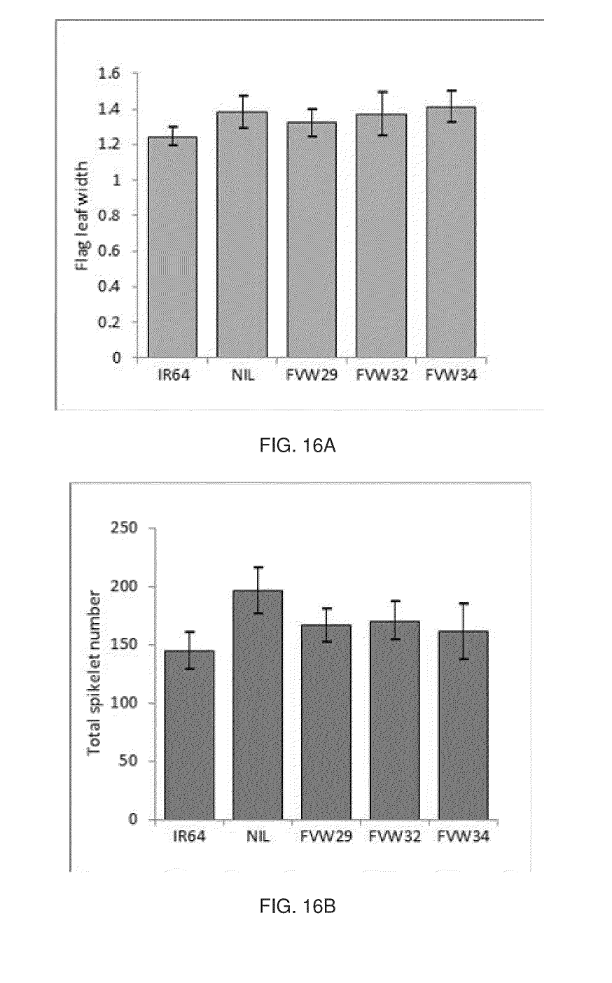

The nal1 (loss-of-function) mutant Fn188 similarly showed reduced TSN and FLW relative to its wild type, Taichung 65 (FIG. 12). These results demonstrate that SPIKE (new allele of Nal1 from tropical japonica) enlarges the panicle and flag leaf in correspondence with expression. Although Nal1 was reported to relate to auxin polar transport, no differences in indoleacetic acid (IAA) biosynthesis or transport between IR64 and NIL-SPIKE (FIG. 13) were observed. The transgenic analysis revealed that SPIKE was identical to Nal1, which affects vein patterning in leaves and polar auxin transport. SPIKE, identified from natural variation, is a new allele from tropical japonica, whereas nail, identified from a mutant line, is a loss-of-function mutation. The nal1 mutant was reduced in TSN compared with wild type, while the new allele from tropical japonica in Nal1 showed increased TSN. The data show that the activity of auxin transport at panicle initiation stage is related to TSN. Through increases in TSN, the grain yield of NIL-SPIKE was increased as a consequence.

Enhancing Grain Yield in Indica Cultivars Through SPIKE

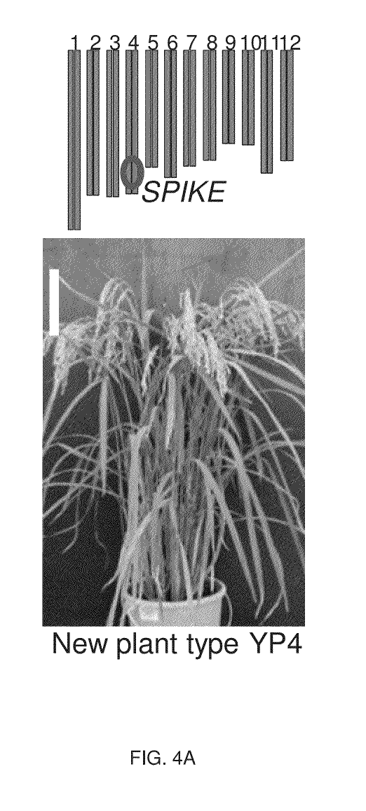

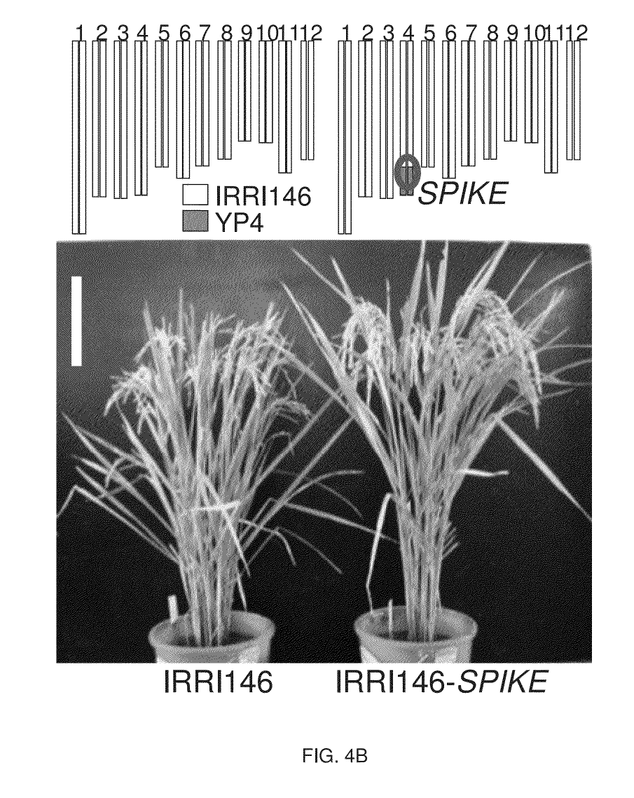

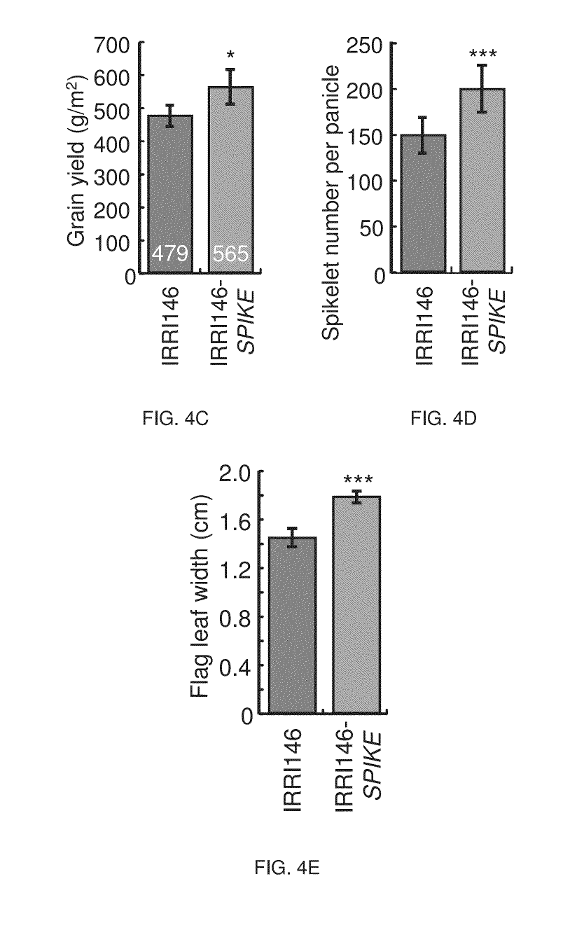

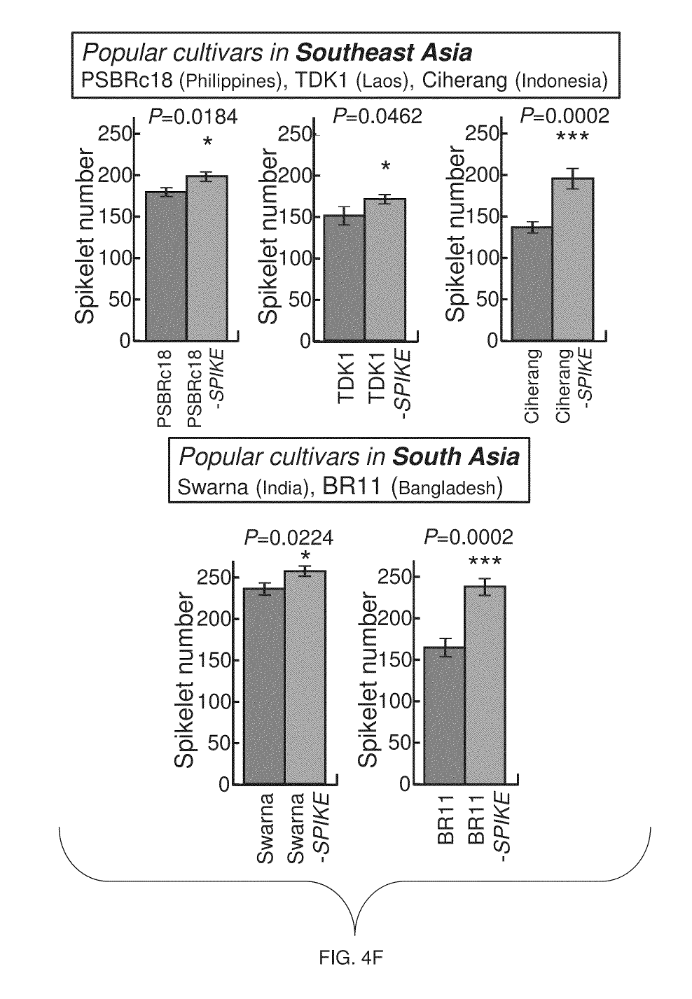

To evaluate the efficacy of SPIKE at increasing yield in different genetic backgrounds, the gene was introgressed it into a new, high-yielding indica cultivar, IRRI146 (released as ‘NSIC Rc158’ in the Philippines). Recurrent backcrossing to IRRI146 and marker-assisted selection (MAS) produced the IRRI146-SPIKE NIL (FIG. 4A, B). As IRRI146-SPIKE has 98% genetic identity to IRRI146, the pleiotropic effects of SPIKE in IRRI146 were similar to those in NIL-SPIKE. GYS, TSN, and FLW of IRRI146-SPIKE were significantly higher than those of IRRI146 (FIG. 4C-E). SPIKE from YP9 was similarly introduced into five popular indica cultivars with different genetic and geographic backgrounds. Its effects were confirmed on the different genetic background of popular indica cultivars, PSBRc18 (IR51672-62-2-1-1-2-3) from Philippines, Ciherang from Indonesia, TDK1 from Laos, BR11 from Bangladesh, Swarna from India. The plants homozygous for SPIKE had significantly higher TSN (FIG. 4F) than the recurrent parent.

Materials and Methods

Plant Materials

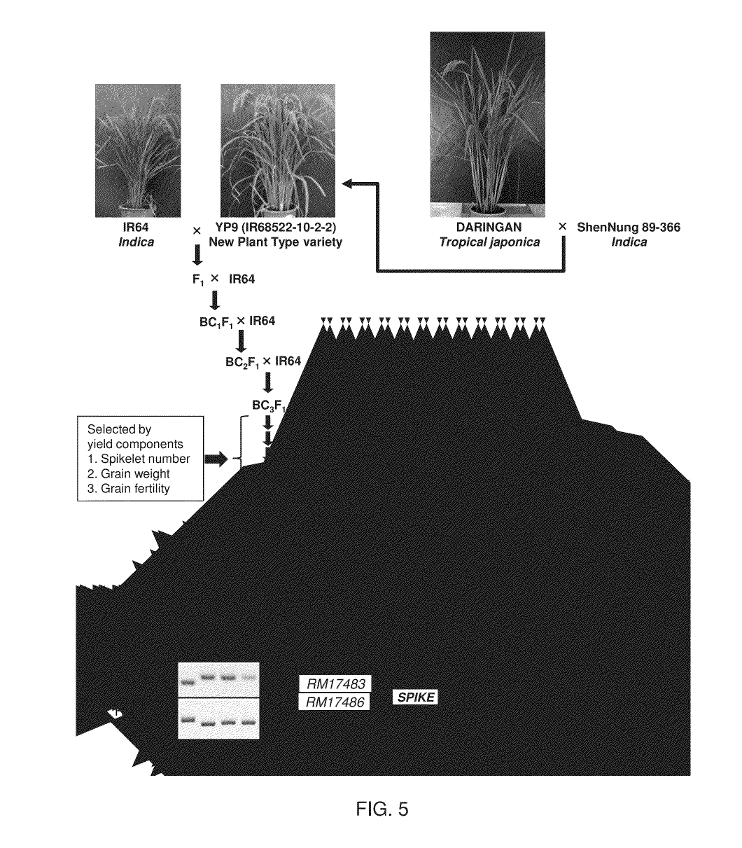

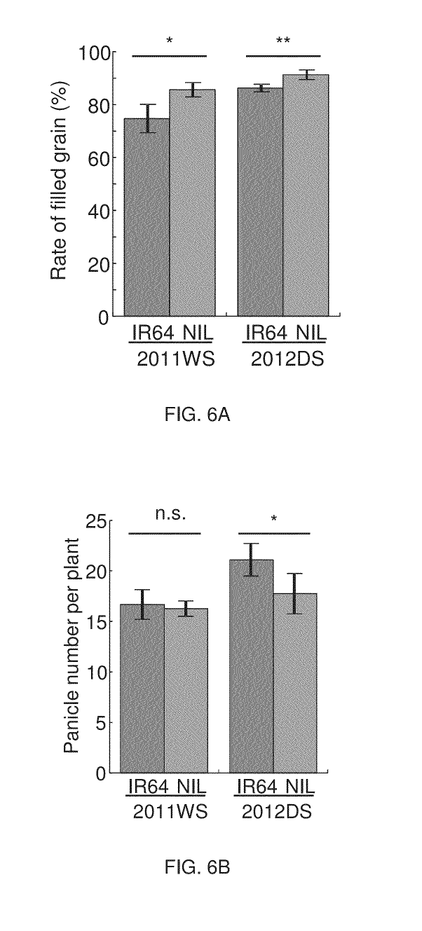

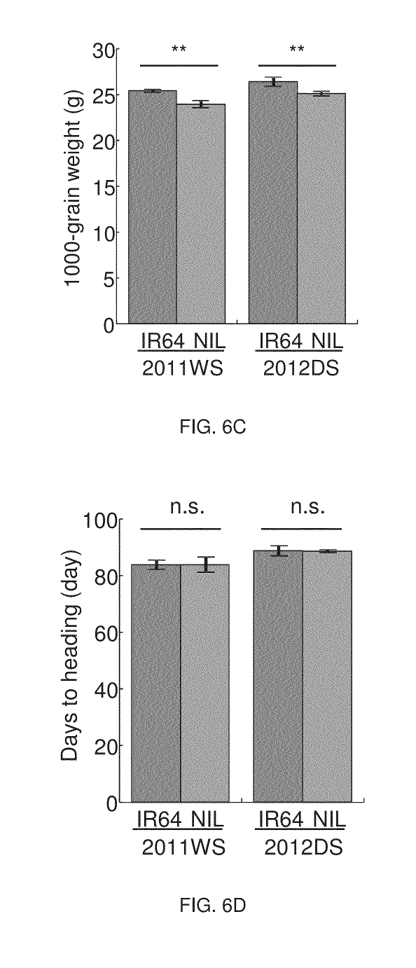

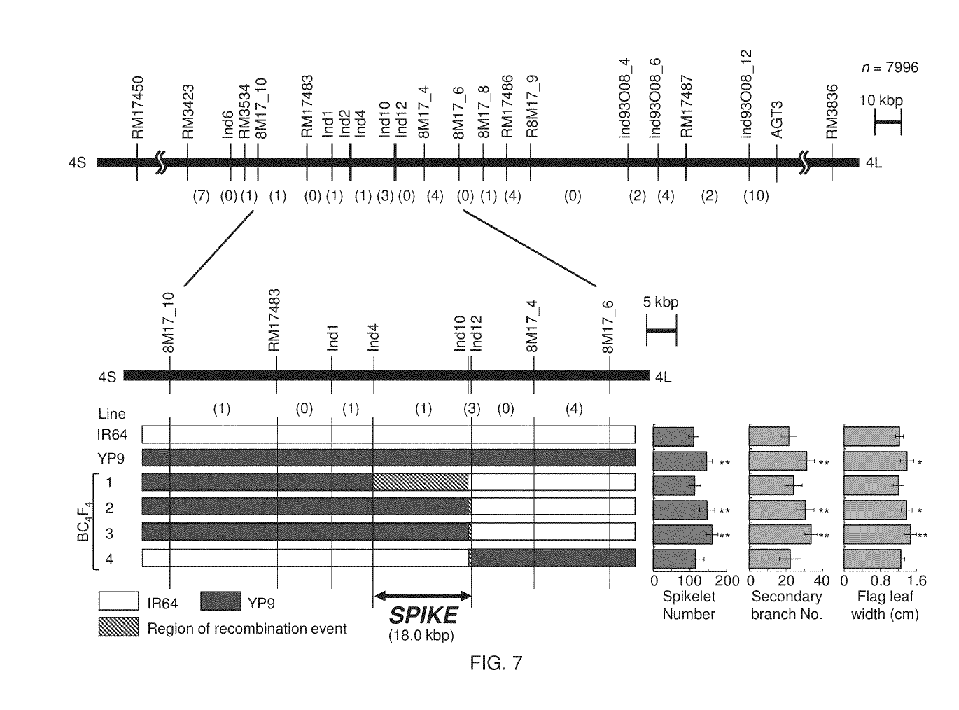

Through backcross breeding, 334 BC₃-derived ILs were developed, which have variation in agronomic traits inherited from NPT cultivars, in the genetic background of indica cultivar IR64. We selected an IL with high TSN: YTH326 (IR84640-11-110-6-4-2-2-4-2-2-3-B), derived from NPT cultivar YP9 (IR68522-10-2-2), which was derived from a cross between indica cultivar Shennung 89-366 and tropical japonica landrace Daringan (FIG. 5). Using a BC₄F₂ population derived from a cross between IR64 and YTH326, qTSN4 was identified, for high TSN, between SSR markers RM3423 and RM17492 on the long arm of chromosome 4. NIL-SPIKE was developed by self-pollination of a plant selected from the BC₄F₂ population and was used for evaluating agronomic traits, transformation, and expression.

Line Fn188, carrying nail, was provided by Kyushu University under the National Bioresource Project. Fn188 had been developed from BC₃ progeny derived from a cross between a nal1 mutant as the donor parent and japonica cultivar Taichung 65 as the recurrent parent. The nal1 locus has been mapped between markers C1100 and C600 on the long arm of chromosome 4. Fn188 was used for agronomic characterization to compare with the effects of SPIKE, since Nal1 was considered to be the same as SPIKE.

Development of IRRI146-SPIKE

A high-yielding indica cultivar, IRRI146 (IR77186-122-2-2-3), has recently been released as ‘NSIC Rc158’ in the Philippines. Progeny of a cross between NPT IR65564-22-2-3 from tropical japonica Bali Ontjer and IRRI146 were backcrossed to IRRI146 three times. In each generation, MAS was conducted using SPIKE-flanking markers RM5503 and RM6909. A whole-genome survey of 96 BC₃F₁ plants using 116 polymorphic SSR markers that covered all chromosomes was conducted. One BC₃F₁ plant was selected and self-pollinated to develop a NIL for SPIKE in the IRRI146 genetic background. This IRRI146-SPIKE was compared with the recurrent parent for agronomic traits and grain yield.

Development of Indica Cultivars with SPIKE

SPIKE was introgressed into five popular cultivars through backcrossing and MAS: PSBRc18 (IR51672-62-2-1-1-2-3) (Philippines), Ciherang (Indonesia), TDK1 (Laos), BR11 (Bangladesh), and Swarna (India). Progeny of the cross between YP9 and each cultivar were backcrossed to the popular cultivar twice. In each generation, MAS was conducted using the SPIKE-flanking markers Ind2 and RM17487. Plants homozygous for SPIKE were selected from each BC₂F₂ population and evaluated for TSN in the field.

Phenotypic Evaluation of SPIKE

All plants were grown in a field at IRRI, Los Baños, Laguna, the Philippines, and evaluated for 1000-grain weight, PN, FLW, and TSN at maturity. The panicle rachis was sectioned at 1 cm below the neck, and VBN were counted under a stereomicroscope. RDW of plants that were grown in pots was measured at maturity.

To evaluate grain yield, IR64, NIL-SPIKE, IRRI146, and IRRI146-SPIKE were grown in a randomized plot with four replications per line. The area of each plot was at least 4.8 m²; three plants were transplanted per hill at 21 days after sowing at 20 cm between hills and 25 cm between rows. As a basal dressing, 30 kg/ha each of N, P, and K were applied the day before transplanting, and 30 kg/ha of N was applied twice as a topdressing at 2 and 4 weeks after transplanting. At maturity, 1.0 m² of rice plants (20 hills in each plot) was harvested, and plants were dried in an oven at 70° C. for 5 days. GYS was calculated on a 14% moisture content basis. Grain chalkiness was evaluated with a Grain Inspector (Cervitec 1625 Grain Inspector, FOSS Analytical, Hillerød, Denmark) with four replications per line.

High-Resolution Linkage Map

The genomic DNA of 7996 BC₄F₃ plants generated from BC₄F₂ plants heterozygous for SPIKE was extracted from fresh leaves. The genomic DNA of 1073 BC₄F₃ plants with recombination between flanking markers RM17450 and RM3836 was individually extracted from freeze-dried leaves by the cetyl trimethylammonium bromide method. 41 BC₄F₃ plants were selected that demonstrated recombination between RM3423 and AGT3 were self-pollinated to generate BC₄F₄ lines to be used for a progeny test. Among the BC₄F₄ lines, homozygous plants from representative recombinants were selected and evaluated for TSN and FLW. Twenty-two DNA markers were used for map construction (Table 1).

TABLE 1
DNA markers used for high-resolution mapping
of QTLs for total spikelet number on chromosome 4.

| | | | SEQ | | SEQ | | Predicted |
|---|---|---|---|---|---|---|---|
| Primer | | Forward primer | ID | Reverse primer | ID | | size |
| application | Marker | sequence (5′-3′) | NO: | sequence (5′-3′) | NO: | (Motif)ₙ | (bp)^{a} |
| DNA | RM17450 | ATCGACAAACCA | 8 | ACTTTGATGAACG | 9 | (AT)₁₉ | 288 |
| markers | | CTCTGCACTCC | | CGGACTCG | | (SEQ | |
| for | | | | | | ID NO: | |
| genotyping | | | | | | 10) | |
| | RM3423 | CAAGAGATCATC | 11 | CCAAACAAATGGC | 12 | (CT)₁₈ | 149 |
| | | ACTGGTACTGG | | CTCAGAT | | (SEQ | |
| | | | | | | ID NO: | |
| | | | | | | 13) | |
| | Ind6 | CTAATTCGGCCC | 14 | CGGGGAAACGAG | 15 | — | 159 |
| | | AACTCTGA | | TATTCA | | | |
| | RM3534 | TTGAGCTTCGTCT | 16 | CAGCTCCCACCAT | 17 | (AG)₁₂ | 129 |
| | | ACAAGCG | | CTCTCTC | | (SEQ | |
| | | | | | | ID NO: | |
| | | | | | | 18) | |
| | 8M17_10 | CCTCCTTCAAGC | 19 | GTCGCTGACACGT | 20 | (GCG)₆ | 128 |
| | | TTCCAACTG | | ACGATACTC | | (SEQ | |
| | | | | | | ID NO: | |
| | | | | | | 21) | |
| | RM17483 | TAGCTTCGGTTCT | 22 | AAACAGATTGCTC | 23 | (AGG)₈ | 148 |
| | | TGATCGTTGG | | ACCACCTTGG | | (SEQ | |
| | | | | | | ID NO: | |
| | | | | | | 24) | |
| | Ind1 | CTTTGGTGGTCA | 25 | TGTTCATCTCCCG | 26 | — | 192 |
| | | TGTGATGC | | TTCTGCT | | | |
| | Ind2 | ACAAGAAGCCGG | 27 | CTCCTCCGGTCCT | 28 | — | 105 |
| | | GAAACCTA | | CCTTAAC | | | |
| | Ind4 | GGTGGTATCTTG | 29 | AACACGAACCCTA | 30 | — | 196 |
| | | TGCCGTCT | | CCCACAC | | | |
| | Ind10 | TTTGGTCGCGTTT | 31 | TTGGAGAACTCCC | 32 | — | 124 |
| | | CTTCC | | TGGTTTG | | | |
| | Ind12 | GATTTTGGGCGC | 33 | ACTGAAGGAAACC | 34 | — | 194 |
| | | ATTGAG | | AGCCAGA | | | |
| | 8M17_4 | ACCAAGAAATCA | 35 | GAGGGAGGAAGA | 36 | (AC)₈ | 150 |
| | | GCGACCAC | | AGATGACG | | (SEQ | |
| | | | | | | ID NO: | |
| | | | | | | 37) | |
| | 8M17_6 | GTGAACGACTTC | 38 | AGGATCCCTCGTC | 39 | (CCA)₅ | 192 |
| | | CCGGAGTT | | CTGGAT | | (SEQ | |
| | | | | | | ID NO: | |
| | | | | | | 40), | |
| | | | | | | (CAC)₅ | |
| | | | | | | (SEQ | |
| | | | | | | ID NO: | |
| | | | | | | 41) | |
| | 8M17_8 | CGCACGATGTGG | 42 | TGAGAGATGAGTG | 43 | (TGG)₆ | 189 |
| | | GATATG | | CCTCACG | | (SEQ | |
| | | | | | | ID NO: | |
| | | | | | | 44) | |
| | RM17486 | TGGAATCACAAA | 45 | CTACCTCAAGCTC | 46 | (AG)₁₆ | 194 |
| | | CCACGACTAGG | | CACGACTTCC | | (SEQ | |
| | | | | | | ID NO: | |
| | | | | | | 47) | |
| | 8M17_9 | GCCATGGAGGTA | 48 | CTGTCAGCCACTC | 49 | (TC)₇ | 106 |
| | | GCAACAGT | | TGATCCA | | (SEQ | |
| | | | | | | ID NO: | |
| | | | | | | 50) | |
| | ind93O08_4 | TCCTCCTCGAGA | 51 | TTTCTTCCAGGCA | 52 | — | 119 |
| | | CCTCTCCT | | CTGAGG | | | |
| | ind93O08_6 | AGAACGGCGACG | 53 | CTACATCACGGAG | 54 | — | 152 |
| | | ACATCTT | | TGGCAGA | | | |
| | RM17487 | CGGAGCATGTGG | 55 | GGAGAGGGCAAG | 56 | (AAG)₇ | 252 |
| | | AGAGGAACTCG | | GGCTTCTTCG | | (SEQ | |
| | | | | | | ID NO: | |
| | | | | | | 57) | |
| | ind93O08-12 | CACAAGCTGCAG | 58 | GGACGAGTCGTAC | 59 | — | 239 |
| | | GACAAGAA | | ACGGTTT | | | |
| | AGT3 | CAAACCGAACCA | 60 | GAGAGAGACGAT | 61 | (CG)₇ | 173 |
| | | CGATACG | | CCCCACAG | | (SEQ | |
| | | | | | | ID NO: | |
| | | | | | | 62) | |
| | RM3836 | ACTGTGGAGTAC | 63 | GAAACGGAAACG | 64 | (GA)₂₂ | 126 |
| | | AGGTCGGC | | AAACCCTC | | (SEQ | |
| | | | | | | ID NO: | |
| | | | | | | 65) | |
| RT-PCR | Ex6.2 | GTGGCAGTGACG | 66 | CTACAGTCGTGAC | 67 | Os04g5 | 1406 |
| | | AATGTATTGG | | GGTGGAAATG | | 2479 | |
| | Ex7.2 | GTCGAAGGATGG | 68 | GGCAGTGTCATAA | 69 | Os04g5 | 586 |
| | | GAGTCAAG | | TCAGTTCCG | | 2500 | |
| | Ex8.1 | ATGAGCTACCAA | 70 | TCAGAAGCACATG | 71 | Os04g5 | 624 |
| | | GGTCCTC | | TCGAGC | | 2504 | |
| | UBQ5 | ACCACTTCGACC | 72 | ACGCCTAAGCCTG | 73 | Os01g2 | 69 |
| | | GCCACTACT | | CTGGTT | | 2490 | |
| | seq8M17-56 | CGCTCAATAGCC | 74 | CCATCACAGTCCC | 75 | Os04g5 | 75 |
| | | TCATAGGG | | AGTTGTG | | 2479 | |
| Ubi::SPIKE1 | 8M17-c1 | ATGAAGCCTTCG | 76 | TCATTTCTCCAGG | 77 | Full- | |
| con- | | GACGATAAGG | | TCAAGGC | | length | |
| struction | | | | | | cDNA | |
| | | | | | | of | |
| | | | | | | SPIKE1 | |
| amiRNA | pRS300A | CTGCAAGGCGAT | 78 | | | miR309a | |
| con- | | TAAGTTGGGTAA | | | | precursor | |
| struction | | C | | | | | |
| | pRS300B | GCGGATAACAAT | 79 | | | miR309a | |
| | | TTCACACAGGAA | | | | precursor | |
| | | ACAG | | | | | |
| | Exon1_I | AGTATAAGAAGT | 80 | — | | amiRN | |
| | miR-s | ATGCTGCGCTAC | | | | A1 | |
| | | AGGAGATTCAGT | | | | | |
| | | TTGA | | | | | |
| | Exon1_II | TGTAGCGCAGCA | 81 | — | | amiRN | |
| | miR-a | TACTTCTTATACT | | | | A1 | |
| | | GCTGCTGCTACA | | | | | |
| | | GCC | | | | | |
| | Exon1_III | CTTAGCGGAGCT | 82 | — | | amiRN | |
| | miR*s | TACTTCTTATATT | | | | A1 | |
| | | CCTGCTGCTAGG | | | | | |
| | | CTG | | | | | |
| | Exon1_IV | AATATAAGAAGT | 83 | — | | amiRN | |
| | miR*a | AAGCTCCGCTAA | | | | A1 | |
| | | GAGAGGCAAAA | | | | | |
| | | GTGAA | | | | | |
| | Exon4_I | AGTTAATATCAA | 84 | — | | amiRN | |
| | miR-s | GTTCCAGACGCC | | | | A4 | |
| | | AGGAGATTCAGT | | | | | |
| | | TTGA | | | | | |
| | Exon4_II | TGGCGTCTGGAA | 85 | — | | amiRN | |
| | miR-a | CTTGATATTAAC | | | | A4 | |
| | | TGCTGCTGCTAC | | | | | |
| | | AGCC | | | | | |
| | Exon4_III | CTGCGTCAGGAT | 86 | — | | amiRN | |
| | miR*s | CTTGATATTAATT | | | | A4 | |
| | | CCTGCTGCTAGG | | | | | |
| | | CTG | | | | | |
| | Exon4_IV | AATTAATATCAA | 87 | — | | amiRN | |
| | miR*a | GATCCTGACGCA | | | | A4 | |
| | | GAGAGGCAAAA | | | | | |
| | | GTGAA | | | | | |
| Promoter | UP6-1 | GCGAATTCTCCG | 88 | GCGGATCCACAGT | 89 | Promoter | |
| SPIKE1 | | AACCAAACACCA | | TTGCGAACCTATT | | region | |
| with GUS | | ACACAC | | ATA | | of | |
| con- | | | | | | SPIKE1 | |
| struction | | | | | | | |

^{a}PCR product size was estimated based on Nipponbare genome sequence.

Transformation of SPIKE

A fragment encompassing the full-length coding region of SPIKE was amplified from cDNA derived from young panicles of NIL-SPIKE using primer pair 8M17-c1. The fragment was ligated into the binary vector pCAMBIA1300int-prUbi1-tNOS between the maize ubiquitin promoter and the nopaline synthase terminator to generate the overexpression vector. Using Agrobacterium-mediated transformation, we introduced the vector into IR64. The regenerated plants were evaluated for transgene copy numbers by Southern blot analysis. For gene silencing of SPIKE, the amiRNA approach was used. Two 21-bp amiRNA sequences—amiRNA1 (TATAAGAAGTATGCTGCGCTA (SEQ ID NO: 4), for the first exon of SPIKE) and amiRNA4 (TTAATATCAAGTTCCAGACGC (SEQ ID NO; 5), for the fourth exon)—were designed using Web MicroRNA Designer 3 software. The amiRNA precursors (Table 1) were generated through site-directed mutagenesis using overlapping PCR with plasmid pNW55 as a template. The precursors were ligated into the binary vector pCAMBIA1300int-prUbi1-tNOS to generate the silencing vectors. Using Agrobacterium-mediated transformation, we introduced the vectors into NIL-SPIKE. The transgenic plants (T₀) were transplanted into pots, and T₁ plants were transplanted in a screenhouse at 20 cm between hills and 30 cm between rows. These plants were evaluated for TSN and FLW.

To generate the promoter:GUS vector, a 1918-bp fragment was amplified upstream from the ATG codon of SPIKE using primer pair UP6-1. The amplified fragment was ligated into the binary vector pCAMBIA0380 (Cambia, Canberra, ACT, Australia) upstream of the GUS reporter gene. This vector was introduced into IR64 by Agrobacterium-mediated transformation. Organs of the regenerated plants were sampled to analyze GUS activity.

Expression Analysis and IAA Transport

Total RNA from each organ was extracted by using an RNeasy Plant Mini Kit (Qiagen, Calif., USA). To identify a candidate gene for SPIKE, RT-PCR was performed using 1 μg of total RNA. PCR was performed using 1 μL of cDNA with the gene-specific primers for each candidate (Table 1). For comparison of expression in different organs, total RNA of young panicle, culm, leaf sheath, leaf, and root was extracted at the panicle initiation stage. RT-PCR was performed with 500 ng of total RNA using primer pair seq8M17-56 and a ReverTra Ace qPCR RT Kit (Toyobo, Osaka, Japan). qRT-PCR reactions were carried out with ⅕ cDNA mixtures using primer pair seq8M17-56 with LightCycler 480 SYBR Green I Master Mix on a LightCycler 480 System (Roche Applied Science). The data were normalized to the expression of a house hold gene, Ubiquitin (Os01g22490).

The rate of IAA biosynthesis in IR64 and NIL-SPIKE coleoptiles was investigated by measuring the amount of IAA that was transported from cut coleoptiles to an agar block (FIG. 13) by gas chromatography—selected ion monitoring—mass spectroscopy (GC-SIM-MS). To investigate polar IAA transport in IR64 and NIL-SPIKE coleoptiles, 3 μM IAA was applied to the top of coleoptile sections (1.5-3.0 mm) for 30 min, then incubated the coleoptiles on an agar block for 10 min, and measured the transported IAA by GC-SIM-MS as above.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1-11. (canceled)

12. A method for producing a rice plant with improved grain yield, the method comprising:
a) providing a first rice plant comprising a gene SPIKE;
b) transferring a nucleic acid encoding gene SPIKE from the first rice plant to a second rice plant;
c) analyzing the second rice plant for the gene SPIKE;
d) identifying and selecting a second rice plant comprising the gene SPIKE and exhibiting improved grain yield when compared to the second rice plant prior to the transfer.

13. The method according to claim 12, wherein the gene SPIKE comprises a polynucleotide sequence selected from the group consisting of: SEQ ID NO: 1; SEQ ID NO: 2; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102.

14. The method according to claim 12, wherein the gene SPIKE comprises a polynucleotide sequence at least 70%, at least 75%, at least 80%, at least 85%, at least 90%, at least 95%, at least 96%, at least 97%, at least 98%, at least 99%, or 100% identical to a sequence selected from the group consisting of: SEQ ID NO: 1; SEQ ID NO: 2; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102.

15-19. (canceled)

20. The method according to claim 12, wherein the transfer of the nucleic acid from the first rice plant to the second rice plant is performed by crossing the first rice plant with the second rice plant to produce offspring plants comprising the gene SPIKE, and wherein steps c) and d) are performed on one or more offspring plants.

21. The method according to claim 12, wherein the transfer of nucleic acid from the first rice plant to the second rice plant is performed by a transgenic method, by crossing, by backcrossing, by protoplast fusion, by a doubled haploid technique, or by embryo rescue.

22. The method according to claim 12, wherein backcrossing results in introgression of the gene SPIKE, and recovery of the second rice plant's genome of at least 85%, at least 87%, at least 90%, at least 92%, at least 94%, at least 96%, or at least 98%.

23-24. (canceled)

25. A rice plant with improved grain yield, comprising the gene SPIKE, and wherein the gene SPIKE is not in the natural genetic background of the rice plant.

26. The method according to claim 12, wherein the first rice plant is selected from an isogenic line of rice plants derived from New Plant Type (NPT) cultivar YP9.

27. The method according to claim 12, wherein the first rice plant is selected from the Oryza sativa subspecies tropical japonica.

28. The method according to claim 12, wherein the first rice plant is Daringan.

29. The method according to claim 12, wherein the second rice plant is selected from the Oryza sativa subspecies indica.

30. The method according to claim 29, wherein the second rice plant is selected from the group consisting of: PSBRc18; Ciherang; TDK1; BR11; and Swarna.

31. A transgenic plant cell comprising:
a) at least one plant promoter; and
b) at least one polynucleotide encoding a polypeptide sequence at least 70% identical to that of a protein SPIKE (SEQ ID NO: 3);
wherein the promoter and polynucleotide are operably linked and incorporated into the plant cell chromosomal DNA.

32. The transgenic plant cell of claim 31, wherein the type of cell is selected from the group consisting of: rice; wheat; sorghum; and maize.

33. (canceled)

34. A transgenic plant comprising a plurality of cells of claim 31.

35-36. (canceled)

37. The transgenic plant of claim 34, wherein the plant is a rice plant.

38. The transgenic plant of claim 34, wherein the polynucleotide sequence is at least 70%, at least 75%, at least 80%, at least 85%, at least 90%, at least 95%, at least 96%, at least 97%, at least 98%, at least 99%, or 100% identical to that of SPIKE.

39. (canceled)

40. A seed of a plant of claim 34.

41. (canceled)

42. The transgenic plant of claim 34, wherein said plant exhibits a phenotype selected from the group consisting of: increased grain yield per m² relative to a corresponding non-transgenic plant; increased total spikelet number per panicle relative to a corresponding non-transgenic plant; and increased flag leaf width relative to a corresponding non-transgenic plant.

43. A method for selecting transgenic plants of claim 34 comprising:
a) screening a population for increased grain yield, wherein plants in the population comprise at least one transgenic plant cell having recombinant DNA incorporated into its chromosomal DNA wherein said recombinant DNA comprises a promoter that is functional in a plant cell and that is functionally linked to an open reading frame of a polynucleotide sequence at least 70% identical to that of SPIKE, wherein individual plants in said population that comprise at least one transgenic plant cell exhibit a grain yield the same as or greater than a grain yield in control plants which do not comprise at least one transgenic plant cell; and
b) selecting from the population one or more plants that exhibit a grain yield greater than the grain yield in control plants which do not comprise at least one transgenic plant cell.

44-63. (canceled)


SEQUENCE LISTING (102 sequences)

SEQ ID NO: 1 - DNA, 17568 bp, Oryza sativa
cttttttttttgcgtttatcttcgctttccttttttttttcttcttccccttttgagatttcgatcgtagcattggttagcgtgttggtggatgtagttaatcaatccccttttaggtgccaaatgtcatggaaagcgagctctttgcccttgtttaaccaagcttttgatcgcagcattcaagaattggctccatctccccgcaaaaaaaaaaagaaagaaaaaggaattggctccatcgattcagtaaaaaaacccgaatgccgcggaagttagcattgctcatccgtggattcgacgctggcacactggtttcttggctagagagaaacaacgatgtgcttgctgtttcagccaccaatttcttcccggcttgtgatttttgccttttgtatctgctttctttttcctcttttgagaaagtacacgattcctttgcctaccttcattttcagtgccgattactaaatttgcagtaggtcctactctctcgccgtggtgttggactatcattgggagtagttgtcatgtggatttatgcccatttgctgcgattaaagagagaatatgtgctacgccttaacgttcatggacctgatttcttgaggtcgctgtaactgttgcctttggagaacaagaggaagagggtgtgagtgtgtggagacactaaccgtggcttttgcagattcctcttatctgttttccttgcatggcagttgtgccatacattgatacagatggatcatattcttcgtacaaccggattggagtatgctcgatctgtcatccgtggagaacccgaaattcagattgcttttgttttgcgtggctctcctttgcagttcttgcgggaacctattcccccaagaatatcatgctactccacatcttgttgtttattctactgattccatcttttcgttcgggccaggccagctagctagccgcaagcgctgactgtcttgatcattgattcctccttccacaataactctaaaagattggaagtacatttgcatgattgatggttttcccgtcgctttcggcattcgttatctacctgtccattagccttcaggatcatgctttctgacttgcttgttctcattcttagggccataacttcagcttctcccatctataataggttcgcaaactgttcagcacaatgaagccttcggacgataaggcgcagctctccggtttggcgcaatcagaagaatcgtcacttgatgtggatcaccagtcatttccttgttctccatcaatccaaccggttgcttctgggtgcacacacacagagaacagcgcagcatacttcttatggccgacatccaacctacagcattgtgcagccgagggacgtgcaaactactttggaaaccttcagaaaggattgttgccaaggcaccctggtcggttgcccaaaggtcagcaagcaaatagcttgcttgacttgatgactataagagctttccatagcaagatattgcggcgttttagcctcgggacagcagtgggattccgcatcaggaaaggggatctaacagatatccctgcaatccttgtctttgttgctcgcaaggttcataagaagtggcttaatccagcacaatgtcttcctgctattcttgaggtgcggaaacttaatctccattgttgtttgaatttggtttgggacggtctaagtttttctgctaaatctttcacttagttggatcattaacagggtccaggaggtgtttggtgtgtaggaataagtcttggctggttggtcttggtcttgtggtgcctttttcctgcttttcagctttctaggacagcatctctactttcagtttggatgctctaggacagttaggacagcagtttgttgagatgctctaggacagtttgttgggatgctctgctctaggacagtttgttgggatgctgacagcaccctcttagactagcatggctgcatggctgctacaagcagctataaatatgtatccaatcctctttgggaggacatggcttttgtgtttagtgaatgaagaaaaccagaaaattgccccatctcgagtgccatcctcttctcaatgagagtaaccattgaaattctaacatctggtatcagagccacactttcctgtaggctaaacatctcttgttccaccccttcccaagctcggttgagctctcagccagcagcagcagcagcagcaccggttgcccctccttcccctttcccttccccctctccacacagcagccccccggaggtgcgccatgtccgacgctcggtctcggcgttcggtcgcctcgagcactcggcgtcagcaggacgccgagctcgccgcagcagaagagcgcaggcgagcaacggctcagaccgctgcagcagcagcgagggcggccagactggcggcagcggagttggcagcagccagggcggaggcggaggcggaggcggcggaagatgcggcacgtgcggtggaggtagaggttgagaccttgcgcagcagcatcaacggctccatcgccggcgacatcaccgctgacagggagcttgaggagctggcaagaggaagggcacgggagcgagcagagcggtgggcagcagcccacctccacggcggcggcggcggcccaagggaccgtgcgcccgccgacgggaacccagacgggcgcgggcgtgccggcggcagcccggagcccgcacgcggccctcgcaggcggcacggctccctctcccctgaccggcgccatggtcaccacggcgtccagacggtggtcagggattttggtcccggcggtgggtggcctaccctcaccaaaaccaactacatcgagtgggccgcggtgatgagggtgaggctccaggtgcggcacatgtgggaggcagtccggtacggtgacgtcgactacgacgaggatcggcgggcactggatgccctcatcgctgcagttccgcccgagatgcagttctcgctttcccagaagcggactgccaaggaggcctgggacgccatcgctgcggcacgcatcggcagcgaccgcgcccgcaagtccacactgcaggcactccgcaaggagtgggagaacctggccttcaagccaggtgaggatgttgatgactttgctctccgtctcaacactctcttgcagaaaatggtgcagtacggcgacgacacctacgacgaggagagagctgtcgagaagctcttccgctgcgtccctgagaagtacaggcagatcgctcgctcgattgaatctctgctggacctctccacgatgtcgatcgaggaggcgttaggtcgcctcaaggtcgtcgatggtgatgagccacggcctctctcggggcccatcaccatcggcgggaagctccatctcactcgggagcagtgggaggcctctcagggtgacgggaggaagggggagtcatcttccccgacaggcggccataagccgcgcaaggcgcggggaggtgtccagctacggtgggcgcgaagacgtgccgagggtggcgcccgcagaggcgcccagggcgttgccaccggcaaccacaagccggcacgagacgacacctgccgcaactgtggcaagcttggccattgggccaaagactgtcgacagccacgacgcggccaggcccacgtcgcacgggtggaggaggaagagccggctctgctcctagctcacgcaagcatcgagctacctccagcggcaccggccgcagcggctttcctccaccttgatgagccgaaggtactcgtctccctctgtgacggctccagcaacgacaaggctgatgggtggtacctcgacaccggcgccacccatcacatgaccggccgacgggagttcttcaccgagttcgactccagcgtccgaggcaccgtcaagttcggggacgcctccggcgtggagatcaagggtgttggctccgtcaccttcaccgccaagtccggtgagcacaggctgctcaccggagtctactacatccccgcgttgaggaattctatcatcagcgtgggacagctggatgagaacggctcacgcgtgttggtcgaggacggactcatgaggatttgggatcgccgtcgtcgccttcttgccaaggtaaccagaggcactaatcgactctacatcctcagcgcgctggtcgcacatccagtttgcctcgccgctcgtcgggacgacgaggcgtggcagtggcacgagcgcttcgggcacctccacttcgaggccctgaagcagctcagtgccaaggagatggtgcgaggcatgccgtgccttgaccacgtggagcagctctgcgacgtctgcgtggtgacaaagcagcggcggctcccctttccccagcagacgagcttccgagccaaggagcggctcgggctcgtgcacggggacttgtgtggcccagtgacaccggccacaccaggaggacgacgttatttcctgctgctcgtcgacgacctctcccgctacatgtgggtgatggtcctcggcagcaagggagaggctgcggacgccatcaggcatgcgcaggctgctgcagaggcggagtgcggccgcaagctgcgcgtgctgcgcaccgacaacggcggcgaattcacggcagctgaattcgcgtcgtactgcgctgatgagggcattcagcgccactacaccgcgccgtacagcccacagcagaacggcgtcgtcgagcggcgcaaccagacggttgtggggatggctcgggccctcctcaagcagagggggatgccggccatcttctggggggaggcggtggtgacggccgcctacatcctcaaccgctcgcctaccaaggccctcgatggcaggacaccgtacgaggcttggcatgggcgcaagccggcggtctcccacctgcgggtcttcggctgcctcgcgttcgccaaggagcttggccacatcggcaagctcgacgacaggagcaccccaggggtgttcatcggctacgcggagggctcgaaggcctaccgcatcctcgacccggagacacagcgtgtgcgcattacgcgcgatgttgtgttcgacgaagggcgagggtgggtatgggacaaggcggtggatgatggttcgactccgacgtacgacgacttcaccatcgagtacgtccacttcgagggagctgggggagtaggcagctcttcttcaccgagcgtgtctaccccagtccccgagcctccaccgactccaacaccaacacaccctcgggccacgacttcgactacgacgagctcttcgtcgacaccaccccagccggtgtccccacacgctccagcaccaacagccactcctccgggcacgtctactccgacgccagctcgtgttgagcgcagcccggtggagttcgttactccgctctcccacgacggggagcgcatcgacgcgtaccatgacggcgagcagctacggtaccgtacgatggaggatcttctcggcgaccagccggtgccgggactggtgcctcgcaacctggaggcgcagttgcaccttgcgtgcgacgacggtgagcctcggtcttttgcagaggccgagaaacacgcggcatggcgtgccgcgatgcagtcggagatggacgcggttcaggagaaccgcacctgggagcttgctgacctccctcgtggtcaccgcgcgatcacccttaagtgggtgttcaagctgaagagggatgaagccggagccatcgtcaagcacaaggctcgcttggtggcacgcggtttcgtgcagcaggaggggatcgactacgacgatgccttcgctcccgtggcacggatggagtccgtgcgactccttcttgcgctggctgctcaggaaggctggggcgtccatcacatggacgtcaagtcggcgtttctaaacggcgacttgaaggaggaggtctacgtgcaccagccgccgggatttgtgatccccggcaaggagggcaaggtgctacgcctgcacaaggccctctacggcttgcggcaggcaccgagggcgtggaatgccaagttggattccacgctcaaggggatgggcttcgagcaaagcccgcacgaggcggccatctaccggcggggcaatggaggaaatgccttgctggtgggtgtctacgtcgacgacttagtgatcaccggcaccaaggatgcagaggtcgcggcgttcaaggaggagatgaaggccaccttccagatgagtgatctggggcctctctccttctacctggggatcgaagtgcaccaggacaactccgggatcacgcttcgacagaccgcctacgccaagcgcgtcgttgagctggctgggctcaccgattgcaacccagctctcactccgatggaggagagactgaagctgagccgcgacagcacagcggaggaggtggatgctacacagtaccgacgtcttgtggggagccttcgctacctcacccacacacggccggacttggccttctccgtcggctacgtcagtcggttcatgcagcgaccgacgacggagcaccagcaggctgtgaagaggatcatccgctacgttgcggggactctcgaccacggtctctactacccgaggtgtcctggcaaggcacacttcgtcgggtacagcgacagcgaccacgccggcgacatcgacaccagcaagagcacgagcgggattctctttttcctcggcgagtgcctcgttagctggcagtcaatcaagcagcaggtggtggccctgtccagctgcgaggccgagtacatggcagcatccgccgcttcgacccaggcgctctggcttgctcgactgcttagtgatctcctcggcagagatactggagcggtggagctcagggtggatagcaagtccgctctggccctggcaaaaaaccccgtttttcacgaacggagcaagcacatccgagtgaggtaccatttcatccgaagctacttggaggaagggagcatcaaggcgagctacatcaacaccaaggaccagcttgcggatttgctcaccaaaccccttgggaggatcaagttccttgagctttgctcccggatcgggatggctcaactctcccacaagacggcgcacaagacttagggggagaatgtaggaataagtcttggctggttggtcttggtcttgtggtgcctttttcctgcttttcagctttctaggacagcatctctactttcagtttggatgctctaggacagttaggacagcagtttgttgagatgctctaggacagtttgttgggatgctctgctctaggacagtttgttgggatgctgacagcaccctcttagactagcatggctgcatggctgctacaagcagctataaatatgtatccaatcctctttgggaggacatggcttttgtgtttagtgaatgaagaaaaccagaaaattgccccatctcgagtgccatcctcttctcaatgagagtaaccattgaaattctaacatctggtatcagagccacactttcctgtaggctaaacatctcttgttccacccctttccaatgtcttcctgctattcttgaggtgcggaaacttaatctccattgttgtttgaatttggtttgggacggtctaagtttttctgctaaatctttcacttagttggatcattaacagggtccaggaggtgtttggtgtgatgttgatgttgttgaattttcgtactacggtgcaccggctcaaacacctaaagagcaaatgttcagtgagcttgttgataagttatgtggcagtgacgaatgtattggttcaggctctcaggtactcgtgttctttccctctctgtgtgtgtgtgtatgtgtgtgtgtttgcaaaaataccatgtgcattaagcacagcatgccatcatctgcacaataattgtacaggacaataccatgtactagcaattgtacccttcatgttgtaaggggaccgttcaaatttgtcaagattcatcgcaagatttgctagtacaaagtggataaattttcatggcactctgaagattatgccaaaaactgaccgttggtttgttggttagtcctaacatctaagtttgaccagttgagcggtggccacagctagtttgaatgctaccaatgttcattacatcaaagcagatgatctgatcggagattggaaggagtttctccttctaaatcatatcatatcatttgagatactgcatactatatgttggttcttgtgcagattaaagcttctggattttacacaatcctcctaccgtttaccagtcaaacctttaacatgttgctgtgctctttttgtgaaatttcaacttagtaataatattttgtgacatgcactcaaactacatactccctccatcccaaaaaaatccaacctaggataggacaacaaatctggacaaacactttgtccagatttgctgtgacctatcctaggttgttcttttttgggacggatggagtacttctgaagcgttcgtccttatagttctgtttcattgtttcaggttgcaagccatgaaacttttggtactttgggtgcaattgtgaaacggcgcactggcaacaagcaggttggtttcctcactaaccatcatgtcgcggttgacttggactaccctaatcagaagatgtttcatccattaccacccaatcttgggcctggcgtttatcttggagctgttgaaagagcaacttctttcatcacagatgacgtttggtatggaatctatgctggaacaaacccaggtagagcagttacaaattagctggtcagggctttatctacaattgtttgtctatttaaaaacaatgtcttaagtaacattaaaagacacttcaactcactgtataaatataaaaaataagtttcctgcttcctagttcccatcattcatattaatgccggattgcattttttactagttagaaacaaatcaacatttagttttgattgtaaaaaaaacatactatgtttgtgcttttctggtcatttaacacatttaccattttattgattattccaaatgatgtatataccagtctaattcccaaacatatatgccgccccattatggatttgcagagacatttgtacgagctgacggtgcatttatcccatttgctgatgactttgacatttccaccgtcacgactgtagttaggggagtcggtgacattggggatgtcaaggttatagatctgcagtgtccgctcaatagcctcatagggaggcaagtatgcaaagttggcagaagctctggtcacacaactgggactgtgatggcctatgcccttgagtacaatgacgagaaaggaatatgcttcttcacagacatccttgttgttggtgagaaccgccaaacatttgatttggaaggtgatagcggaagccttattatcctgactagccaagatggtgagaagccgcgtccaattggaattatatggggtggcacagcaaatcgtgggaggttgaagcttacaagtgatcatggccctgaaaactggactagtggggttgatcttggccgtctactcgaccgtctggaacttgatattatcattaccaatgaatcactccaaggttgattcttttccctctattcatttgcattcatcttcagatattccagaaatacgttggactatccaatttacttgatgttttcatactactttgtattgaatctgtacacttttgctccattacaaagctgacagaatttgcctactataaagatgccgtgcagcagcaaagatttgctttggtggccgccgttacctcagctgttggggagtcttccggggtgcctgtcgccatcccggaagagaagatcgaagagatcttcgagccattggggatccaaatccagcaactgcctcgccatgacgtggcggcctctggaactgaaggggaggaggcatccaacacggtggtcaatgtggaagagcaccagttcatctcaaacttcgtcggtatgtcgcccgtgcgcgacgaccaagacgctccgaggagcatcaccaacctgaacaacccctccgaggaagaactcgccatgtcgctccatctgggtgaccgagagcccaagcggctccgttcggactccggatcaagccttgacctggagaaatgaagtgcaaaaagtgctggttctgaatttctgattctggttcttcagagtttattgtcattgtggacagccaaaagtagtagtagatcaaaagacctcatatgcagttcagaaaaattgaacctgcagtaagcttcggtgagcttgaatggctttggcaagcatatcatgtaacatgatgtattattattactacaattgattcagccccttgtaggatgttaggtgctagtcctgcagttttgctgtgctcatgatcgtgagacctgatatacaccattgccggatcccttcctctttgtagaagaagaagaacaatgtgcagggaattatgataatgatgatctttgatctatttccccctattattatttaccaattatcaggtttcaattatcaatcagtctgaaatcttgcatttgcatttgcattttgtcaaccagtctttatcttattaactcgggatgatctgttgcactgtcactgacatgtggggcccctaccccgcgccgcgtcgcgacatacctaaccgtattaaactagatcacctccgagtcttttgttacaaaaggaccagctaccctcagtcaatgactaggtgggaccggaaagtgtgggccactcctgtcagtgacacagaggaagaggcgtaagtacggagccgcgtagccgtcgctcgcttcatccggtttggcgtacggatacgggattgcgaatcccccacgcatccggaaatcggaatcccacccggttcgggaaaaggataaaaccaccccggttcgggaagattcgtccatggctgccctgtcgtccacgcgaccgaagaagcaaggcaagccctagccgccactcccgatttgcttcccagcccttgcggctcctgcgattcgatttggcgggaaattgctcgcgctctcgctcgcgccatcggttcttgaatcggagggattgggagcgggggggaaggtttcttgctttgcagccgaatcttgactggtccagtttcgtgacttacctagtgcccattcttcccgtttgagatttagaagcaagctttggatgaaaagcatcatctttagcttctttgatttgttgggtgaagggggctctcaattctttcgagccattgctgctcattcgagagctatcctttcaccggttggattggaggagctagtccataaatttgtataggaatctgaggaaagggcgctctttacggtgcccatggctgtgctggaggatttgatcagggcgattgagctctggctgcggattgctaaggagcaggtacccttggtggatccaacccttgacccggttctgcttgtccccggcataggcggctcgattcttgaagccgttgatgaggccgggaagaaggagagggtgtgggtgcgcatcctcgctgcggatcatgagtttcgagcacatctctggtccaagtttgatgcttccactggtatgaaaaaaaggagaaaaagaactgataaatgcgatgcctgttctgacttatgaatagtgtacacttagctgttttacatttatttgttagaaatatcaagtagagtagagaatggaaattggaatgcttcatatattagaatgggtggtatactttgataaattgcagggtctttcagtaagacgcagaagctgaaaatatatgataattagaggacaaacatctagagatttatatcttgtttgcgtagacaattagggaactccatatggttttgttgcctttaatttagttcttaaacaactgctagatggtcttcacggatggcctgctgattttccaccttcattcctttgtactgattcagtatagtattctctttctgtgtcatttgattccaaattattctcatggagcagggaaaactgtctctgtcgatgagaaaacaaatattgttgtccctgaggatagatatggactatacgctattgacactctggaccccgacatggtaaaacattatttcatatatttggttaaagctctaccttttactgttattaatttccaaaggttcctttaaccatcgattatattcaattctgtttaacttctgtctattaccttccactccgcgactgccatatcagtattgtttctatttagtgattccttgccggaaacattcacagaccatcctattcttgttaaaatagccactagtgatgattttgtgtattgtctttctgatggctgaacaataacttgcaagtgcttacactctttaaagccaatttattgtgaacctgtaccttcacagctcaacaaacgcaaatcatatactgcagttgcatacctgatctgcaccacttatattacaatttgaattagcagatcattggcgatgatagtgtttgctactatcacgacatgatagtgcaaatgatcaaatggggataccaagaggggaaaacattatttggatttggttatgatttccgccagagtaacaggtgaattgtgcacttctattcctgttttgcacatacagttctgtttatactttacaattctttagtgtttgctcaactgtgctattataattatttatgaacacacttgtgaagttgatcattctttctgttggtagaagtgagttaacagttctctttttaattctcctttggctcaggctttcagaaacacttgatagattttccagaaagttggagtcagtttacatagcttccggagaaaaaaagatcaatctcattactcattcaatgggaggattgcttgtcaaatgcttcatgtccctccatagtgatgtgggtatttctctgtatattttggatctttacctgagatttgtcatgctcagcatcttcgctacctgaaactgcaaggctataatgttgctccctttctataggtcttcgagaaatacataaagagttggattgcaattgctgcaccatttcaaggtactgccttttggggccccttaatttttaaagaaagaaatagggagattatcacatatttgtgtgttttagtattacatgatgattatattctgtacatgataatctattaccttcaatgtatgggagcaactttacatggcaaacaacttgctactctatgacaggtgcacctgggtacataactactagtctgctgaatggtatgtcttttgtcgaaggatgggagtcaagattctttatttccaagtggagtatgcagcaattggtatgttttggtagccatacattgatcctgtcctcaaatatgttcatgcagatgacattacttttgtcttacaaacaaagactgaaactgtttcttttttttgcttcagttgctcgaatgcccatcaatttacgaattgttggctaactcgaccttccaatgggaagatactccatatctgcagatctggagacagaaattggatactaatggcaagaaaagtgccatgttagagtcatatgagccagatgaagcaataaaaatgattagagaagctctttccaagcatgaggcaagtcattttatttatacatttacattgcaatataatttacccaatataataagctgttttcgaaaaaaaaatgatgttggtatattgcagatcatttctgatggtatgcacattccattgccccttgatatggatatattgagatgggcaaaagagacacaggatgttttgtgcaatgcaaagcttccaaaatcagtgaagttctacaatatttacggaactgattatgacactgcccataccgttcggtaattttcctatctggacacctgtaattttagtttatcttttgcttccagaccaatcagaatctggcattggttcaacttaattagatgggctcatgaatttatgtacataaatggctcaatagataggaacaaatgagaacttcatgcttacatcatgttagtagtgcttaactcaagttcaaaattgcttgttgcaatgcaattgagtgcttctctctctatccattgtgcaaagtaggatatggttgtaaagatgtgtttccatttaaatgcagcaaggaactatgcagtcaagttaaactaaagatttttatgttgatagaaatatcccccaaaggcatgataacaccatctctgcatggtgtttactacataactaaaagtttagtactgtatgtgtcaagttactaatacccacatctttcgcagctacgggagtgaacaccatccaatttcaaatctcagtgacctcttgtatactcaggttagttcaattggtaaatgctactatgaataccaattttgtggctctgtggagtacaaaataatcccactggttttgtgcgttaactatgttttacttcattcagggcaactacatctgtgttgatggtgatggatctgtccctgtagaatcagcaaaggtatgatattattagtgttttggttgaaaatttctttcaaaaagaaaaaatgaatcaggagaaaagtttacattgagaaaattgagaaccatcaacgactttgcaggcagatggcctcgatgcagtggcaagagttggggttgctgcagaccaccgaggaatcgtctgtgatcgtcacgtgttccggataattcagcactggctccatgccggtgagcctgacccattctacgatcccctcaacgactacgtcatactcccaacagccttcgagatcgagaagtaccacgagaaacacggggatatcacatcggttagagaggactgggagatcatctcccatcgcgatgacgaaagcaagaggccagccgagcttcctcctatgttcaacacgctatcggcgtcccgcgagggtgaagacggctcgctggaagaggcgcaggcgacgatctttgttcatccagagagcaaagggaggcagcatgtggaagttagggcagttggagtcacccatgacggctagtcaagccagtcatacgaaaacacacggttgtcaactagctagtctgcacactccaaagcaaagtggacaatgtaaatataagacgtccctagctatgaactacgtgtaattttgctgccttgtaaataccagaactgaaaatatactgccactggatgatgatacgaatagaaaggagaaagaaaaggatgaacttgatatgttaaaattgtatcaggtgcttgaaatgttgttactggaaactgatcatcctgactcggttgatcaagcgatgagacctccgagttatcattgtcttatgcatctttctagtccttgtttaattcatcttaagcttgttggctatgtcaaatctttgtttgggcattgcagtttcttgctgcgctcgtcatctcttaagcaaaaaggtcaaatttgttgcaaatagtctgcagtacttcgccgttttttccaatggacaggaatgtgtcctgatatttggcatccttgtccgtgccgtgcattgatttttgaggttgcaattttcattgacaaacatacagtagaattaatccaatcaataaaaattagtgtgattcataatcattaatccgatcaataaaatttagggtgattcgtatagtactaaaatcgaaatcattgataaacagaagtgtcccagattacgatgaatgatacaaaatggatccacttttgaatttgacaacacatgaaatccaaatgatccaagaacagtaactacataatactagatgcacacatctcaccacatacttaaaaaatctcgatcatgacacagcacaaatgatgtgaaaagacgatcacaagctgctctataagtagagtatcacatcatccttgatgccatcttctcatcagaagcacatgtcgagcaggcagcagcagcagagagcagcacaactgcaaaaaaaatcacatcgacagagacagaagctttagaggcaagaacactcacaattatcctgataaaaaatggatcgatgttttgatcttctcttttggtgtgtggtttctttcttaattaccatcctttcaagaagccgtcgccgcggctctgcgagtcctgcggcggcggcggcggctgctgctgatacccgccgtagtcggcggcgactggcggcggcgcgccgtacgccggcggtgggtaccccgtccccggtggcgggtacgctgcaacagtagataatcacacatgcatcagctgatgaatttgtccatcaaaacgaactcagaaaaaaaaataaaaaatgtcgggatgcatgtattttctcctaatcttaataaaatttggtcgcgtttcttcccgaccaggcaaaaaaaaaaagaaacaaagaaaaaaaaaggagacctgacacacaactctcacaattattttctctgtgctaaatcacaaaccagggagttctccaatcagatcaagaaaccaagagatttcaatacctgcagtgccgggaggaggaccttggtagctcatgatcactactactcaatagtagtaaatcctccaaagagcaagaagagatgcaaaccaagaacaagaatgcaagataatggagatgagaaatccaagctctcctcagtttatttaacgctggactctctatctcctagctatatatactcctacatatatagtgctactaaactttatttatttccttagccgcgttaataattaattaatgacaagaagaaaaagaacgccgaatggctgctgcttatcccactcttctgaaaaacgcgttgttttacgtcgtttacgctacatttatttagacgtacggcatcgggttctccatctcttctacatgcctttttcatttttcaaattgaaaaatattgaaaaggcgtcgtttttagattgtttgaacatgcactatgtgtctatatgtactgccttttcagttttcaaatgttgatcagagatggcggggaactgaagaaggctcgtttgtcaatttgacagaatcttcttctaagcaatgcagggataagctagctaacttaacctctaagctttccattattccaaaatagtcatgcaatgaggagtagttcgttcgattcccactgctctagctggtttagggcctgggaatagcaggttcgtcagcttgcatttggatgattatggttgagaaagagtctgcttcgagttcatcaccatttcgaactgctccactacgatcaggttaattccacagttgagcgaacgtccaaacgaatcgtgttctatcggttaggtgaagattttgatcgaaatattgtcttttatgtgctatttttgcatagaaaatccttttggcatgcagctatagcgccaataaattcttgctgcaggtttccttcagctttgtcctttttctttcctggtcaaagttggttgcctgtgaaccgtgtcgttggtgttcataatcttcagtagtgcttcaccaaccatagtccaataggcaattttgggcgatattttggtaagctgaaagatattttaaattgagctagaac

SEQ ID NO: 2 - DNA, 2168 bp, Oryza sativa
cgctttcggcattcgttatctacctgtccattagccttcaggatcatgctttctgacttgcttgttctcattcttagggccataacttcagcttctcccatctataataggttcgcaaactgttcagcacaatgaagccttcggacgataaggcgcagctctcaggtttggcgcaatcagaagaatcgtcacttgatgtggatcaccagtcatttccttgttctccatcaatccaaccggttgcttctgggtgcacacacacagagaacagcgcagcatacttcttatggccgacatccaacctacagcattgtgcagccgagggacgtgcaaactactttggaaaccttcagaaaggattgttgccaaggcaccctggtcggttgcccaaaggtcagcaagcaaatagcttgcttgacttgatgactataagagctttccatagcaagatattgcggcgttttagcctcgggacagcagtgggattccgcatcaggaaaggggatctaacagatatccctgcaatccttgtctttgttgctcgcaaggttcataagaagtggcttaatccagcacaatgtcttcctgctattcttgagggtccaggaggtgtttggtgtgatgttgatgttgttgaattttcgtactacggtgcaccggctcaaacacctaaagagcaaatgttcagtgagcttgttgataagttatgtggcagtgacgaatgtattggttcaggctctcaggttgcaagccatgaaacttttggtactttgggtgcaattgtgaaacggcgcactggcaacaagcaggttggtttcctcactaaccatcatgtcgcggttgacttggactaccctaatcagaagatgtttcatccattaccacccaatcttgggcctggcgtttatcttggagctgttgaaagagcaacttctttcatcacagatgacgtttggtatggaatctatgctggaacaaacccagagacatttgtacgagctgacggtgcatttatcccatttgctgatgactttgacatttccaccgtcacgactgtagttaggggagtcggtgacattggggatgtcaaggttatagatctgcagtgtccgctcaatagcctcatagggaggcaagtatgcaaagttggcagaagctctggtcacacaactgggactgtgatggcctatgcccttgagtacaatgacgagaaaggaatatgcttcttcacagacatccttgttgttggtgagaaccgccaaacatttgatttggaaggtgatagcggaagccttattatcctgactagccaagatggtgagaagccgcgtccaattggaattatatggggtggcacagcaaatcgtgggaggttgaagcttacaagtgatcatggccctgaaaactggactagtggggttgatcttggccgtctactcgaccgtctggaacttgatattatcattaccaatgaatcactccaagatgccgtgcagcagcaaagatttgctttggtggccgccgttacctcagctgttggggagtcttccggggtgcctgtcgccatcccggaagagaagatcgaagagatcttcgagccattggggatccaaatccagcaactgcctcgccatgacgtggcggcctctggaactgaaggggaggaggcatccaacacggtggtcaatgtggaagagcaccagttcatctcaaacttcgtcggtatgtcgcccgtgcgcgacgaccaagacgctccgaggagcatcaccaacctgaacaacccctccgaggaagaactcgccatgtcgctccatctgggtgaccgagagcccaagcggctccgttcggactccggatcaagccttgacctggagaaatgaagtgcaaaaagtgctggttctgaatttctgattctggttcttcagagtttattgtcattgtggacagccaaaagtagtagtagatcaaaagacctcatatgcagttcagaaaaattgaacctgcagtaagcttcggtgagcttgaatggctttggcaagcatatcatgtaacatgatgtattattattactacaattgattcagccccttgtaggatgttaggtgctagtcctgcagttttgctgtgctcatgatcgtgagacctgatatacaccattgccggatccc

SEQ ID NO: 3 - protein, 582 aa, Oryza sativa
MKPSDDKAQLSGLAQSEESSLDVDHQSFPCSPSIQPVASGCTHTENSAAYFLWPTSNLQHCAAEGRANYFGNLQKGLLPRHPGRLPKGQQANSLLDLMTIRAFHSKILRRFSLGTAVGFRIRKGDLTDIPAILVFVARKVHKKWLNPAQCLPAILEGPGGVWCDVDVVEFSYYGAPAQTPKEQMFSELVDKLCGSDECIGSGSQVASHETFGTLGAIVKRRTGNKQVGFLTNHHVAVDLDYPNQKMFHPLPPNLGPGVYLGAVERATSFITDDVWYGIYAGTNPETFVRADGAFIPFADDFDISTVTTVVRGVGDIGDVKVIDLQCPLNSLIGRQVCKVGRSSGHTTGTVMAYALEYNDEKGICFFTDILVVGENRQTFDLEGDSGSLIILTSQDGEKPRPIGIIWGGTANRGRLKLTSDHGPENWTSGVDLGRLLDRLELDIIITNESLQDAVQQQRFALVAAVTSAVGESSGVPVAIPEEKIEEIFEPLGIQIQQLPRHDVAASGTEGEEASNTVVNVEEHQFISNFVGMSPVRDDQDAPRSITNLNNPSEEELAMSLHLGDREPKRLRSDSGSSLDLEK

SEQ ID NO: 4 - DNA, 21 bp, Artificial Sequence
  Description of Artificial Sequence Synthetic oligonucleotide
tataagaagtatgctgcgcta

SEQ ID NO: 5 - DNA, 21 bp, Artificial Sequence
  Description of Artificial Sequence Synthetic oligonucleotide
ttaatatcaagttccagacgc

SEQ ID NO: 6 - protein, 582 aa, Oryza sativa
MKPSDDKAQLSGLAQSEESSLDVDHQSFPCSPSIQPVASGCTHTENSAAYFLWPTSNLQHCAAEGRANYFGNLQKGLLPRHPGRLPKGQQANSLLDLMTIRAFHSKILRRFSLGTAVGFRIRKGDLTDIPAILVFVARKVHKKWLNPAQCLPAILEGPGGVWCDVDVVEFSYYGAPAQTPKEQMFSELVDKLCGSDECIGSGSQVASHETFGTLGAIVKRRTGNKQVGFLTNRHVAVDLDYPNQKMFHPLPPNLGPGVYLGAVERATSFITDDVWYGIYAGTNPETFVRADGAFIPFADDFDISTVTTVVRGVGDIGDVKVIDLQCPLNSLIGRQVCKVGRSSGHTTGTVMAYALEYNDEKGICFFTDILVVGENRQTFDLEGDSGSLIILTSQDGEKPRPIGIIWGGTANRGRLKLTSDHGPENWTSGVDLGRLLDRLELDIIITNESLQDAVQQQRFALVAAVTSAVGESSGAPVAIPEEKVEEIFEPLGIQIQQLPRHDVAASGTEGEEASNTVVNVEEHQFISNFVGMSPVRDDQDAPRSITNLNNPSEEELAMSLHLGDREPKRLRSDSGSSLDLEK

SEQ ID NO: 7 - protein, 582 aa, Oryza sativa; identical to SEQ ID NO: 3

SEQ ID NO: 8 - DNA, 23 bp, Artificial Sequence
  Description of Artificial Sequence Synthetic primer
atcgacaaaccactctgcactcc

SEQ ID NO: 9 - DNA, 21 bp, Artificial Sequence
  Description of Artificial Sequence Synthetic primer
actttgatgaacgcggactcg

SEQ ID NO: 10 - DNA, 38 bp, Artificial Sequence
  Description of Artificial Sequence Synthetic oligonucleotide
atatatatatatatatatatatatatatatatatatat

SEQ ID NO: 11 - DNA, 23 bp, Artificial Sequence
  Description of Artificial Sequence Synthetic primer
caagagatcatcactggtactgg

SEQ ID NO: 12 - DNA, 20 bp, Artificial Sequence
  Description of Artificial Sequence Synthetic primer
ccaaacaaatggcctcagat

SEQ ID NO: 13 - DNA, 36 bp, Artificial Sequence
  Description of Artificial Sequence Synthetic oligonucleotide
ctctctctctctctctctctctctctctctctctct

SEQ ID NO: 14 - DNA, 20 bp, Artificial Sequence
  Description of Artificial Sequence Synthetic primer
ctaattcggcccaactctga

SEQ ID NO: 15 - DNA, 18 bp, Artificial Sequence
  Description of Artificial Sequence Synthetic primer
cggggaaacgagtattca

SEQ ID NO: 16 - DNA, 20 bp, Artificial Sequence
  Description of Artificial Sequence Synthetic primer
ttgagcttcgtctacaagcg

SEQ ID NO: 17 - DNA, 20 bp, Artificial Sequence
  Description of Artificial Sequence Synthetic primer
cagctcccaccatctctctc

SEQ ID NO: 18 - DNA, 24 bp, Artificial Sequence
  Description of Artificial Sequence Synthetic oligonucleotide
agagagagagagagagagagagag

SEQ ID NO: 19 - DNA, 21 bp, Artificial Sequence
  Description of Artificial Sequence Synthetic primer
cctccttcaagcttccaactg

SEQ ID NO: 20 - DNA, 22 bp, Artificial Sequence
  Description of Artificial Sequence Synthetic primer
gtcgctgacacgtacgatactc

SEQ ID NO: 21 - DNA, 18 bp, Artificial Sequence
  Description of Artificial Sequence Synthetic oligonucleotide
gcggcggcggcggcggcg

SEQ ID NO: 22 - DNA, 23 bp, Artificial Sequence
  Description of Artificial Sequence Synthetic primer
tagcttcggttcttgatcgttgg

SEQ ID NO: 23 - DNA, 23 bp, Artificial Sequence
  Description of Artificial Sequence Synthetic primer
aaacagattgctcaccaccttgg

SEQ ID NO: 24 - DNA, 24 bp, Artificial Sequence
  Description of Artificial Sequence Synthetic oligonucleotide
aggaggaggaggaggaggaggagg

SEQ ID NO: 25 - DNA, 20 bp, Artificial Sequence
  Description of Artificial Sequence Synthetic primer
ctttggtggtcatgtgatgc

SEQ ID NO: 26 - DNA, 20 bp, Artificial Sequence
  Description of Artificial Sequence Synthetic primer
tgttcatctcccgttctgct

SEQ ID NO: 27 - DNA, 20 bp, Artificial Sequence
  Description of Artificial Sequence Synthetic primer
acaagaagccgggaaaccta

SEQ ID NO: 28 - DNA, 20 bp, Artificial Sequence
  Description of Artificial Sequence Synthetic primer
ctcctccggtcctccttaac

SEQ ID NO: 29 - DNA, 20 bp, Artificial Sequence
  Description of Artificial Sequence Synthetic primer
ggtggtatcttgtgccgtct

SEQ ID NO: 30 - DNA, 20 bp, Artificial Sequence
  Description of Artificial Sequence Synthetic primer
aacacgaaccctacccacac

SEQ ID NO: 31 - DNA, 18 bp, Artificial Sequence
  Description of Artificial Sequence Synthetic primer
tttggtcgcgtttcttcc

SEQ ID NO: 32 - DNA, 20 bp, Artificial Sequence
  Description of Artificial Sequence Synthetic primer
ttggagaactccctggtttg

SEQ ID NO: 33 - DNA, 18 bp, Artificial Sequence
  Description of Artificial Sequence Synthetic primer
gattttgggcgcattgag

SEQ ID NO: 34 - DNA, 20 bp, Artificial Sequence
  Description of Artificial Sequence Synthetic primer
actgaaggaaaccagccaga

SEQ ID NO: 35 - DNA, 20 bp, Artificial Sequence
  Description of Artificial Sequence Synthetic primer
accaagaaatcagcgaccac

SEQ ID NO: 36 - DNA, 20 bp, Artificial Sequence
  Description of Artificial Sequence Synthetic primer
gagggaggaagaagatgacg

SEQ ID NO: 37 - DNA, 16 bp, Artificial Sequence
  Description of Artificial Sequence Synthetic oligonucleotide
acacacacacacacac

SEQ ID NO: 38 - DNA, 20 bp, Artificial Sequence
  Description of Artificial Sequence Synthetic primer
gtgaacgacttcccggagtt

SEQ ID NO: 39 - DNA, 19 bp, Artificial Sequence
  Description of Artificial Sequence Synthetic primer
aggatccctcgtcctggat

SEQ ID NO: 40 - DNA, 15 bp, Artificial Sequence
  Description of Artificial Sequence Synthetic oligonucleotide
ccaccaccaccacca

SEQ ID NO: 41 - DNA, 15 bp, Artificial Sequence
  Description of Artificial Sequence Synthetic oligonucleotide
caccaccaccaccac

SEQ ID NO: 42 - DNA, 18 bp, Artificial Sequence
  Description of Artificial Sequence Synthetic primer
cgcacgatgtgggatatg

SEQ ID NO: 43 - DNA, 20 bp, Artificial Sequence
  Description of Artificial Sequence Synthetic primer
tgagagatgagtgcctcacg

SEQ ID NO: 44 - DNA, 18 bp, Artificial Sequence
  Description of Artificial Sequence Synthetic oligonucleotide
tggtggtggtggtggtgg

SEQ ID NO: 45 - DNA, 23 bp, Artificial Sequence
  Description of Artificial Sequence Synthetic primer
tggaatcacaaaccacgactagg

SEQ ID NO: 46 - DNA, 23 bp, Artificial Sequence
  Description of Artificial Sequence Synthetic primer
ctacctcaagctccacgacttcc

SEQ ID NO: 47 - DNA, 32 bp, Artificial Sequence
  Description of Artificial Sequence Synthetic oligonucleotide
agagagagagagagagagagagagagagagag

SEQ ID NO: 48 - DNA, 20 bp, Artificial Sequence
  Description of Artificial Sequence Synthetic primer
gccatggaggtagcaacagt

SEQ ID NO: 49 - DNA, 20 bp, Artificial Sequence
  Description of Artificial Sequence Synthetic primer
ctgtcagccactctgatcca

SEQ ID NO: 50 - DNA, 14 bp, Artificial Sequence
  Description of Artificial Sequence Synthetic oligonucleotide
tctctctctctctc

SEQ ID NO: 51 - DNA, 20 bp, Artificial Sequence
  Description of Artificial Sequence Synthetic primer
tcctcctcgagacctctcct

SEQ ID NO: 52 - DNA, 19 bp, Artificial Sequence
  Description of Artificial Sequence Synthetic primer
tttcttccaggcactgagg

SEQ ID NO: 53 - DNA, 19 bp, Artificial Sequence
  Description of Artificial Sequence Synthetic primer
agaacggcgacgacatctt

SEQ ID NO: 54 - DNA, 20 bp, Artificial Sequence
  Description of Artificial Sequence Synthetic primer
ctacatcacggagtggcaga

SEQ ID NO: 55 - DNA, 23 bp, Artificial Sequence
  Description of Artificial Sequence Synthetic primer
cggagcatgtggagaggaactcg

SEQ ID NO: 56 - DNA, 22 bp, Artificial Sequence
  Description of Artificial Sequence Synthetic primer
ggagagggcaagggcttcttcg

SEQ ID NO: 57 - DNA, 21 bp, Artificial Sequence
  Description of Artificial Sequence Synthetic oligonucleotide
aagaagaagaagaagaagaag

SEQ ID NO: 58 - DNA, 20 bp, Artificial Sequence
  Description of Artificial Sequence Synthetic primer
cacaagctgcaggacaagaa

SEQ ID NO: 59 - DNA, 20 bp, Artificial Sequence
  Description of Artificial Sequence Synthetic primer
ggacgagtcgtacacggttt

SEQ ID NO: 60 - DNA, 19 bp, Artificial Sequence
  Description of Artificial Sequence Synthetic primer
caaaccgaaccacgatacg

SEQ ID NO: 61 - DNA, 20 bp, Artificial Sequence
  Description of Artificial Sequence Synthetic primer
gagagagacgatccccacag

SEQ ID NO: 62 - DNA, 14 bp, Artificial Sequence
  Description of Artificial Sequence Synthetic oligonucleotide
cgcgcgcgcgcgcg

SEQ ID NO: 63 - DNA, 20 bp, Artificial Sequence
  Description of Artificial Sequence Synthetic primer
actgtggagtacaggtcggc

SEQ ID NO: 64 - DNA, 20 bp, Artificial Sequence
  Description of Artificial Sequence Synthetic primer
gaaacggaaacgaaaccctc

SEQ ID NO: 65 - DNA, 44 bp, Artificial Sequence
  Description of Artificial Sequence Synthetic oligonucleotide
gagagagagagagagagagagagagagagagagagagagagaga

SEQ ID NO: 66 - DNA, 22 bp, Artificial Sequence
  Description of Artificial Sequence Synthetic primer
gtggcagtgacgaatgtattgg

SEQ ID NO: 67 - DNA, 23 bp, Artificial Sequence
  Description of Artificial Sequence Synthetic primer
ctacagtcgtgacggtggaaatg

SEQ ID NO: 68 - DNA, 20 bp, Artificial Sequence
  Description of Artificial Sequence Synthetic primer
gtcgaaggatgggagtcaag

SEQ ID NO: 69 - DNA, 22 bp, Artificial Sequence
  Description of Artificial Sequence Synthetic primer
ggcagtgtcataatcagttccg

SEQ ID NO: 70 - DNA, 19 bp, Artificial Sequence
  Description of Artificial Sequence Synthetic primer
atgagctaccaaggtcctc

SEQ ID NO: 71 - DNA, 19 bp, Artificial Sequence
  Description of Artificial Sequence Synthetic primer
tcagaagcacatgtcgagc

SEQ ID NO: 72 - DNA, 21 bp, Artificial Sequence
  Description of Artificial Sequence Synthetic primer
accacttcgaccgccactact

SEQ ID NO: 73 - DNA, 19 bp, Artificial Sequence
  Description of Artificial Sequence Synthetic primer
acgcctaagcctgctggtt

SEQ ID NO: 74 - DNA, 20 bp, Artificial Sequence
  Description of Artificial Sequence Synthetic primer
cgctcaatagcctcataggg

SEQ ID NO: 75 - DNA, 20 bp, Artificial Sequence
  Description of Artificial Sequence Synthetic primer
ccatcacagtcccagttgtg

SEQ ID NO: 76 - DNA, 22 bp, Artificial Sequence
  Description of Artificial Sequence Synthetic primer
atgaagccttcggacgataagg

SEQ ID NO: 77 - DNA, 20 bp, Artificial Sequence
  Description of Artificial Sequence Synthetic primer
tcatttctccaggtcaaggc

SEQ ID NO: 78 - DNA, 25 bp, Artificial Sequence
  Description of Artificial Sequence Synthetic primer
ctgcaaggcgattaagttgggtaac

SEQ ID NO: 79 - DNA, 28 bp, Artificial Sequence
  Description of Artificial Sequence Synthetic primer
gcggataacaatttcacacaggaaacag

SEQ ID NO: 80 - DNA, 40 bp, Artificial Sequence
  Description of Artificial Sequence Synthetic primer
agtataagaagtatgctgcgctacaggagattcagtttga

SEQ ID NO: 81 - DNA, 40 bp, Artificial Sequence
  Description of Artificial Sequence Synthetic primer
tgtagcgcagcatacttcttatactgctgctgctacagcc

SEQ ID NO: 82 - DNA, 40 bp, Artificial Sequence
  Description of Artificial Sequence Synthetic primer
cttagcggagcttacttcttatattcctgctgctaggctg

SEQ ID NO: 83 - DNA, 40 bp, Artificial Sequence
  Description of Artificial Sequence Synthetic primer
aatataagaagtaagctccgctaagagaggcaaaagtgaa

SEQ ID NO: 84 - DNA, 40 bp, Artificial Sequence
  Description of Artificial Sequence Synthetic primer
agttaatatcaagttccagacgccaggagattcagtttga

SEQ ID NO: 85 - DNA, 40 bp, Artificial Sequence
  Description of Artificial Sequence Synthetic primer
tggcgtctggaacttgatattaactgctgctgctacagcc

SEQ ID NO: 86 - DNA, 40 bp, Artificial Sequence
  Description of Artificial Sequence Synthetic primer
ctgcgtcaggatcttgatattaattcctgctgctaggctg

SEQ ID NO: 87 - DNA, 40 bp, Artificial Sequence
  Description of Artificial Sequence Synthetic primer
aattaatatcaagatcctgacgcagagaggcaaaagtgaa

SEQ ID NO: 88 - DNA, 30 bp, Artificial Sequence
  Description of Artificial Sequence Synthetic primer
gcgaattctccgaaccaaacaccaacacac

SEQ ID NO: 89 - DNA, 29 bp, Artificial Sequence
  Description of Artificial Sequence Synthetic primer
gcggatccacagtttgcgaacctattata

SEQ ID NO: 90 - protein, 639 aa, Brachypodium distachyon
MKPSDDRMQLLGLTQSEESSLDVEGYCYHNETFPCSPSMQPIASGCVHTENSAAYFLWPTSNLQHCAAEGRANYFGNLQKGLLPVLPGKLPKGQQANSLLDLMTVRAFHSKILRRFSLGTAVGFRIKKGVLTDIPAIIVFVARKVHKKWLNPNQCLPAILAGPGGVWCDVDVVEFSYYGAPAQTPKEQMFSELVNKLCGSDEYIGSGSQVASQDTFGTLGAIVKRRTNNRQVGFLTNRHVAVDLDYPNQKMFHPLPPNLGPGVYLGAVERATSFITDDVWYGIYAGTNPETFVRADGAFIPFADDFDISTVTTIVREVGEIGDVKVIDLQCPINSLIGRQVCKVGRSSGHTTGTVMAYALEYNDEKGICFFTDLLVVGENRQTFDLEGDSGSLILLTSQDGEKPLPIGIIWGGTANRGRIKLTSDHGPENWTTGVDLGRLLDRLELDLIITNESLKDAVQQHRNALVAAVISAVGESSTVAATAPEEKAEEVFEPLGIKIQQLPRHDVTISATEGEDTANTSADVEEHQFISNFGSMSPARRDQDTPRNIGNLNNPSEEELTMSLHVGDREPKRLRSDAESNLDLEKRPRLDPESSLDLEKGPRSDPEASLDLEKRPPPDREPSLDLEKQPRSDPEARV

SEQ ID NO: 91 - protein, 662 aa, Triticum sp.
MRPSDDRMQLSGLTQSEESSLDVEGHCSHHEAFPCSPSMQPVASGCVHTENSAAYFLWPTSNLQHCAAEGRANYFGNLQKGLLPILPGKLPKGQQANSLLDLMTIRAFHSKILKRFSLGTAVGFRITKGVLTETPAILVFVARKVHKKWLNPNQCLPAILAGPGGVWCDVDVVEFSYYGAPAQTPKEQMFSELVNKLCGSDEYIGSGSQVASQDTFGTLGAIVKRRTNNKQVGFLTNRHVAVDLDYPNQKMFHPLPPNLGPGVYLGAVERATSFITDDVWYGIYAGTNPETFVRADGAFIPFADDFDISTVTTIVREVGEIGDVKIIDLQCPIKSLIGRQVCKVGRSSGHTTGTVMAYALEYNDEKGICFFTDLLVVGENRQTFDLEGDSGSLIILTSQDGEKPRPIGIIWGGTANRGRIKLTSEYGPENWTTGVDLGRLLDRLELDLIINDESLKDAVQEQRKAFVAAINSAIGESSAVTVTAPEATPAEKVEEIFEPLGIQIQQLPRHDLASATTEGEGAANTPSDMEERQFISNFVGMSPVRRDHDARRSIANLNNPSEEELAMSLHLGDREPKRLRLDPESSLDLEKQPHPDPEPSLDLEERPRPDPEMMSLDLEKQPRPDPEPGLDLEKQPRSDPEPGLDLEKRLPADPEPSIDLEK

SEQ ID NO: 92 - protein, 607 aa, Sorghum bicolor
MRPSDDRAQLSGFAQSDESTLDVEGHCYHQQSFPCSPSMQPIASGCTHTENSAAYFLWPTSNLQHCAAEGRANYFANLSKGLLPKSGKLPKGQQANSLLDLMTIRAFHSKILRCFSLGTAVGFRIRKGVLTDIPAILCFVARKVHKKWLNPTQCLPAIVEGPGGIWCDVDVVEFSYYGAPAQTPKEQMFTELVDKLCGSDECIGSGSQVLAKIDLNYLKVADKDSWNDAMAVASQDTFGTLGAIVKRRTGNKQIGFLTNRHVAVDLDYPNQKMYHPLPPNLGPGVYLGAVERATSFITDDVWYGIYAGTNPETFVRADGAFIPFAHDFDISTVSTTVRGVGDIGDVKFIDLQCPLNSLIGRQVCKIGRSSGHTTGTVMAYALEYNDEKGISFFTDLLVVGENRQTFDLEGDSGSLIILTGQDSEKPRPIGIIWGGTANRGRLKLRCDHGPENWTSGVDLGRLLDRLELDLIITSESLKDAVQQQRLAMVAAANSAVGESSTAAVPVPEEKVEELYEPLGIKIEQLPRHDVSASGTEGEEAAVVNVEERQFISNFVGMSPVRGDQDAPRQIANLNNPSEEELAMSLHLGDREPKRLRTDTESDLDLEK

SEQ ID NO: 93 - protein, 582 aa, Zea mays
MRPSDGRTQLSGFAQSDESTLDVEGHCYHQQSFPSSPSMQPIASGCTHTENSAAYFLWPTSNLQHCAAEGRANYFANLSKGLLPKSGRLPKGQQANSLLDLMTIRAFHSKVLRCFSLGTAVGFRIRKGALTDIPAILCFVARKVHKKWLNPDQCLPAIVEGPGGIWCDVDVVEFSYYGAPAQNPKVQMFTELVDKLCGSDECIGSGSQVASQDTFGTLGAIVKRRTGNKQIGFLTNRHVAVDLDYPNQKMYHPLPPNLGPGVYLGAVERATSFITDDVWYGIYAGTNPETFVRADGAFIPFAHDFDISTVTTTVRGVGDIGDVKVIDLQSPLNSLIGRQVCKIGRSSGHTTGTVVAYALEYNDEKGISFFTDLLVVGENRQTFDLEGDSGSLIILTGQDNEKPCPIGIIWGGTANRGRLKLRCDHGPENWTSGVDLGRLLDRLELDLIITNESLKDAVQQQRLALVAAANSAVGESSTAAVPAPEEKVEIFEPLGIKIEQLPRHDVSATTEGDEAAVINVEERQFISNFVGMSPVRDDQDAPRQIANLNNPSEEELAMSLHLGDREAKRLRTDTESELDLEK

SEQ ID NO: 94 - DNA, 1749 bp, Oryza sativa
atgaagccttcggacgataaggcgcagctctccggtttggcgcaatcagaagaatcgtcacttgatgtggatcaccagtcatttccttgttctccatcaatccaaccggttgcttctgggtgcacacacacagagaacagcgcagcatacttcttatggccgacatccaacctacagcattgtgcagccgagggacgtgcaaactactttggaaaccttcagaaaggattgttgccaaggcaccctggtcggttgcccaaaggtcagcaagcaaatagcttgcttgacttgatgactataagagctttccatagcaagatattgcggcgttttagcctcgggacagcagtgggattccgcatcaggaaaggggatctaacagatatccctgcaatccttgtctttgttgctcgcaaggttcataagaagtggcttaatccagcacaatgtcttcctgctattcttgagggtccaggaggtgtttggtgtgatgttgatgttgttgaattttcgtactacggtgcaccggctcaaacacctaaagagcaaatgttcagtgagcttgttgataagttatgtggcagtgacgaatgtattggttcaggctctcaggttgcaagccatgaaacttttggtactttgggtgcaattgtgaaacggcgcactggcaacaagcaggttggtttcctcactaaccatcatgtcgcggttgacttggactaccctaatcagaagatgtttcatccattaccacccaatcttgggcctggcgtttatcttggagctgttgaaagagcaacttctttcatcacagatgacgtttggtatggaatctatgctggaacaaacccagagacatttgtacgagctgacggtgcatttatcccatttgctgatgactttgacatttccaccgtcacgactgtagttaggggagtcggtgacattggggatgtcaaggttatagatctgcagtgtccgctcaatagcctcatagggaggcaagtatgcaaagttggcagaagctctggtcacacaactgggactgtgatggcctatgcccttgagtacaatgacgagaaaggaatatgcttcttcacagacatccttgttgttggtgagaaccgccaaacatttgatttggaaggtgatagcggaagccttattatcctgactagccaagatggtgagaagccgcgtccaattggaattatatggggtggcacagcaaatcgtgggaggttgaagcttacaagtgatcatggccctgaaaactggactagtggggttgatcttggccgtctactcgaccgtctggaacttgatattatcattaccaatgaatcactccaagatgccgtgcagcagcaaagatttgctttggtggccgccgttacctcagctgttggggagtcttccggggtgcctgtcgccatcccggaagagaagatcgaagagatcttcgagccattggggatccaaatccagcaactgcctcgccatgacgtggcggcctctggaactgaaggggaggaggcatccaacacggtggtcaatgtggaagagcaccagttcatctcaaacttcgtcggtatgtcgcccgtgcgcgacgaccaagacgctccgaggagcatcaccaacctgaacaacccctccgaggaagaactcgccatgtcgctccatctgggtgaccgagagcccaagcggctccgttcggactccggatcaagccttgacctggagaaatga

SEQ ID NO: 95 - DNA, 1749 bp, Oryza sativa; identical to SEQ ID NO: 94

SEQ ID NO: 96 - DNA, 1749 bp, Oryza sativa; identical to SEQ ID NO: 94

SEQ ID NO: 97 - DNA, 1749 bp, Oryza sativa; identical to SEQ ID NO: 94

SEQ ID NO: 98 - DNA, 1749 bp, Oryza sativa; identical to SEQ ID NO: 94

SEQ ID NO: 99 - DNA, 1749 bp, Oryza sativa; identical to SEQ ID NO: 94

SEQ ID NO: 100 - DNA, 1749 bp, Oryza sativa; identical to SEQ ID NO: 94

SEQ ID NO: 101 - DNA, 1749 bp, Oryza sativa; identical to SEQ ID NO: 94

SEQ ID NO: 102 - DNA, 1749 bp, Oryza sativa; identical to SEQ ID NO: 94